(19) United States
(12) Patent Application Publication
(10) Pub. No.: US 2024/0110926 A1
(43) Pub. Date: Apr. 4, 2024

(54) TNF-ALPHA AND IL-1ALPHA POSITIVE COPD TREATMENT WITH ANTIBIOTICS

(71) Applicant: GLAXOSMITHKLINE BIOLOGICALS SA, Rixensart (BE)

(72) Inventors: Sonia BUDRONI, Siena (IT); Simona RONDINI, Siena (IT); Silvia ROSSI PACCANI, Siena (IT)

(73) Assignee: GLAXOSMITHKLINE BIOLOGICALS SA, Rixensart (BE)

(21) Appl. No.: 18/257,412

(22) PCT Filed: Dec. 15, 2021
(86) PCT No.: PCT/EP2021/085961

§ 371 Date: Jun. 14, 2023

(30) Foreign Application Priority Data
Dec. 18, 2020 (GB) 2020083.8

(51) Int. Cl. G01N 33/68; A61K 45/00

(52) U.S. Cl. CPC G01N 33/6869; A61K 45/00; G01N 2333/525; G01N 2333/545; G01N 2800/122; G01N 2800/26; G01N 2800/52

(57) ABSTRACT

The present invention relates to antibiotic agents for use in the treatment of patients presenting with an exacerbation of chronic obstructive pulmonary disease (COPD). More particularly the present invention relates to antibiotic agents for use in the treatment of COPD exacerbations in patients that, through analysis, have been identified as having an increased concentration of the IL-1α and TNF-α cytokine biomarkers in a biological sample taken from said patient. The present invention is further directed to methods of diagnosing and methods of treating a patient presenting with an exacerbation of COPD, said methods including identifying 1 the patient is suffering from an exacerbation that is associated with a bacterial infection.

### TECHNICAL FIELD OF THE INVENTION

The present invention relates to antibiotic agents for use in the treatment of an exacerbation of COPD, particularly a subgroup of patients that are experiencing an exacerbation of COPD that have an increased concentration of IL-1α and TNF-α in a biological sample taken from said patients. The present invention is further directed to methods for treating a subject experiencing an exacerbation of COPD, the method including identifying that a patient is experiencing an exacerbation of COPD that is associated with a bacterial exacerbation if the concentration of IL-1α and TNF-α are increased and administering the antibiotic agent to the subject.

### BACKGROUND TO THE INVENTION

Chronic Obstructive Pulmonary Disease (COPD), a leading cause of morbidity and mortality worldwide, is characterised by persistent airflow limitation that is typically progressive and associated with an enhanced chronic inflammatory response in the airways to noxious particles or gases. Symptoms of COPD include dyspnea, chronic cough or the production of sputum and spirometry analysis is typically required to confirm diagnosis. A post-bronchodilator FEV₁/FVC<0.70 confirms the presence of airflow limitation and thus COPD. Several factors have been identified that influence the development and progression of COPD, the most studied of which is smoking tobacco cigarettes. Other factors include for example infection, genetics and exposure to particles, such as occupational dusts, chemical agents and pollution from heating fuels.

COPD can be mild to very severe and assessment of the disease is necessary for administration of the appropriate medication to control and/or relieve symptoms. An assessment of a patient's symptoms may include, for example, the COPD Assessment Test (CAT), the COPD Control Questionnaire (CCQ), the St. George's Respiratory Questionnaire (SGRQ) test, Forced Expiratory Volume (FEV₁) analysis, and an assessment of their exacerbation risk.

A number of medications are available for the treatment of COPD and these are used mainly to reduce symptoms and reduce the frequency and severity of exacerbations and can include bronchodilators, such as beta₂-agonists and anticholinergics, corticosteroids (oral and inhaled), PDE-4 inhibitors, methylxanthines and combinations of some of the above. Bronchodilator medications are vital to symptom management in COPD and the choice between monotherapy with a beta₂-agonist, anticholinergic or theophylline, or combination therapy is dependent upon how effective the medication controls a patient's symptoms.

According to the 2014 Global Initiative for Chronic Obstructive Lung Disease (GOLD) strategy, for COPD patients with a high risk of exacerbations, inhaled corticosteroids are recommended in combination with a beta₂-agonist or anticholinergic. However, exacerbations of COPD remain a major risk for patients suffering with COPD and is a major cause of COPD related hospitalisations and mortality. Improved treatment options are sought.

More than 80% of exacerbations of COPD are managed on an outpatient basis with pharmacological therapies including bronchodilators, corticosteroids and antibiotics. The GOLD strategy outlines three classifications of COPD exacerbations. Mild exacerbations (which are treated with short acting bronchodilators [SABDs] only), Moderate (treated with SABDs plus antibiotics and/or oral corticosteroids) or severe exacerbations which generally requires hospitalization.

Exacerbations may be caused by several different factors, for example by smoking or due to inflammation. Bafadehl and colleagues performed an extensive inflammatory profiling study and identified multiple subgroups of exacerbation (eosinophil-predominant, bacteria/neutrophil-dependent, viral-mediated, or pauci-inflammatory [Bafadhel M et al., Am J Respir Crit Care Med Vol 184. pp 662-671 (2011)]. Personalising the choice of treatment that is administered to a patient with an exacerbation of COPD, based on what subgroup of exacerbation they are suffering from, may lead to improved outcomes.

However, the clinician is not always aware of the cause of an exacerbation when they treat a patient's symptoms, and thus the choice of treatment may be sub-optimal. For example, if a patient is treated with an antibiotic but the exacerbation is not associated with a bacterial infection, they have likely been treated using a sub-optimal therapeutic approach (e.g. they may have derived more benefit with a corticosteroid). Furthermore, they may have been unnecessarily prescribed an antibiotic which may promote antibiotic resistance (AMR).

There is interest in personalised medicine for COPD patents through the identification of biomarkers that can be used to select a particular population of COPD patients that will derive most benefit from a particular pharmacologic treatment. Despite the research that has been performed in this area to date, there exists a need for further, improved therapies for use in the treatment of COPD patients who are presenting with an exacerbation of their COPD symptoms. Thus, there is a further need for therapies for use in the treatment of exacerbations of COPD in individual members of the subpopulation of COPD patients.

### SUMMARY OF THE INVENTION

The inventors of the present application have discovered that by measuring the concentration of cytokine biomarkers in a biological sample taken from a patient presenting with symptoms of an exacerbation of COPD, it can be elucidated whether their exacerbation is associated with a bacterial infection. More particularly, it was discovered that an increased concentration of IL-1α and TNF-α is indicative that the patient is suffering from an exacerbation of COPD that is associated with a bacterial infection. This knowledge can be used by the clinician in their decision regarding whether (or not) to administer an antibiotic agent to the patient. The invention thus provides a rapid, point-of-care assessment to distinguish a bacterial exacerbation of COPD from other causes, for use in a routine clinical care setting.

Thus, in a first aspect of the invention there is provided an antibiotic agent for use in the treatment of an exacerbation of chronic obstructive pulmonary disease (COPD) in a subject, wherein the subject has an increased concentration of IL-1α and TNF-α in a biological sample taken from the subject.

In a second aspect of the invention there is provided, an antibiotic agent for use in a method of treating an exacerbation of COPD in a subject, wherein the method comprises: (a) measuring the concentration of IL-1α and TNF-α in a biological sample taken from the subject, (b) differentiating that the subject is suffering from an exacerbation of COPD that is associated with a bacterial infection if the concentration of IL-1α and TNF-α are increased and, (c) administering the antibiotic agent to the subject.

In a third aspect of the invention there is provided a method of treating an exacerbation of COPD in a subject comprising the steps of: (a) measuring the concentration of IL-1α and TNF-α in a biological sample taken from the subject, (b) differentiating that the subject is suffering from an exacerbation of COPD that is associated with a bacterial infection if the concentration of IL-1α and TNF-α are increased and, (c) administering a therapeutically effective amount of an antibiotic agent to the subject.

In a fourth aspect of the invention there is provided a method for diagnosing that a subject is suffering from an exacerbation of COPD that is associated with a bacterial infection the method comprising: (a) measuring the concentration of IL-1α and TNF-α in a biological sample taken from the subject and, (b) detecting that the level of IL-1α and TNF-α are increased.

In a fifth aspect of the invention, there is provided the use of an antibiotic agent for the manufacture of a medicament for the treatment of an exacerbation of COPD in a subject, wherein the subject has an increased concentration of IL-1α and TNF-α in a biological sample taken from the subject.

In a sixth aspect of the invention there is provided the use of an antibiotic agent for the manufacture of a medicament for use in a method of treating an exacerbation of COPD in a subject wherein the method comprises measuring the concentration of IL-1α and TNF-α in a biological sample taken from the subject, differentiating that the subject is suffering from an exacerbation of COPD associated with a bacterial infection if the concentration of IL-1α and TNF-α are increased and then administering the antibiotic agent to the subject.

### BRIEF DESCRIPTION OF FIGURES

FIG. 1: Raw data distributions. In each panel the horizontal axis is the concentration (pg/ml) of a given cytokine (in log 10 scale) and the y-axis is the count (# of observations per interval of concentration). Dashed lines represent the Lower Limit and the Upper Limit of the quantification range.

FIG. 2: Density distribution of cytokine concentration in Stable states (STs) and in Exacerbation states (EXs).

FIG. 3: To compare the EMMs between disease states, for each cytokine the geometric mean ratio (GMRs) and two-sided 95% CIs by exponentiation (base 10) of the contrast of those EMMs were calculated. Results show that during exacerbations the level of IL-17A, TNF-α, IL-1β and IL-10 significantly increase (>2-fold) compared to the level measured in stable state.

FIG. 4: Results from the mixed model for each cytokine: bars represent the exponential (base 10) 95% CIs of the Estimated Marginal Means (EMMs) in each group (ST or EX).

FIG. 5: Odds ratios from the logistic generalized linear model. Only for IL10 and IL17A did a significant positive effect exist.

FIG. 6: To evaluate differences between type of infection (B, V, E and Pauci) associated with state of disease (ST or EX states), for each cytokine i we applied a linear mixed effect model (the output of which is shown in FIG. 6). For each CTK, the result from the model is shown by horizontal bars which outline the 95% confidence intervals for the Estimated Marginal Means (EMMs) in each group. A clear and significant separation can be measured only for exacerbations associated with bacterial infections (group B EX in the figure) in IL-17A, TNF-α, IL-1β and IL-1α.

FIG. 7: Receiver operating characteristic curves illustrating biomarkers that may positively predict bacteria-associated exacerbations. The area under the curve (AUC) and the 95% confidence interval is shown in the legend.

FIG. 8: The analysis conducted in FIG. 6 was repeated on a follow-up clinical study to evaluate differences between type of infection (Bacterial [B], Viral [V] or None) associated with state of disease (ST or EX states), for each cytokine. For each CTK, the result from the model is shown by horizontal bars which outline the 95% confidence intervals for the Estimated Marginal Means (EMMs) in each group.

### DETAILED DESCRIPTION OF THE INVENTION

### Definitions

As used herein, the term “antibiotic” or “antibiotic agent” refers to an antimicrobial substance active against bacteria. The substance may inhibit the growth and/or replication of a bacterium or kill bacteria outright. Antibiotic agents are medicaments which target bacterial infections within (or on) the body. The main types of antibiotics include: Penicillin (e.g. phenoxymethylpenicillin, flucloxacillin and amoxicillin), Cephalosporins (e.g. cefaclor, cefadroxil and cefalexin), Tetracyclines (e.g. tetracycline, doxycycline and lymecycline); Aminoglycosides (e.g. gentamicin and tobramycin), Macrolides (e.g. erythromycin, azithromycin and clarithromycin), Clindamycin, Sulfonamides and trimethoprim (e.g. co-trimoxazole), Metronidazole and tinidazole, Quinolones (e.g. ciprofloxacin, levofloxacin and norfloxacin), Nitrofurantoin.

As used herein, the term “corticosteroid” means a synthetic pharmaceutical medicament that mimics the action of naturally occurring corticosteroids. Corticosteroids are most often used to treat diseases of immunity and inflammation. Examples of oral corticosteroids include bethamethasone, prednisone, prednisolone, triamcinolone, methylprednisolone, dexamethasone and fludrocortisone. Systemic oral and injectable corticosteroids include glucocorticoids (e.g. hydrocortisone, cortisone, ethamethasoneb, prednisone, prednisolone, triamcinolone and dexamethasone) or mineralocorticoids (e.g. fludrocortisone). Examples of inhaled corticosteroids include beclomethasone dipropionate, budesonide, ciclesonide, flunisolide, fluticasone propionate, fluticasone furoate, or mometasone furoate. Oral and injectable corticosteroids act systemically and are referred to herein as “systemic corticosteroids”, distinguishing them from inhaled corticosteroids, which act topically.

As used herein, the term “exacerbation of COPD” is an event characterised by a worsening of the patient's respiratory symptoms that is beyond normal day-to-day variations. Typically, an exacerbation of COPD leads to a change in medication.

As used herein, the term “increased concentration” refers either a) to a concentration of biomarker in a test biological sample (e.g. a sputum sample) that is higher than the concentration of that biomarker in a reference biological sample (e.g. a sputum sample) which is used as a reference (the reference concentration) or b) to a concentration of a biomarker in a test biological sample (e.g. a sputum sample) that is higher than a defined threshold concentration. The increase in concentration may be measured in pg/ml or may be referred to by a fold-increase in concentration.

As used herein, the term “reference concentration” refers to the concentration of a biomarker in a biological sample which is used as a reference. The reference concentration may be, (i) the concentration in a biological sample obtained from a healthy subject (i.e. a subject which does not have COPD), (ii) the concentration in a biological sample from a subject with COPD which does not have an exacerbation (e.g. when the subject is at a stable state), or (iii) the concentration in a biological sample obtained from the same subject at a different timepoint, for example when the subject is not experiencing an exacerbation of COPD. The reference concentration may be measured in pg/ml. Reference concentration may also refer to a concentration (or concentration range) which has been assigned as being a “normal” concentration or concentration range.

As used herein, the term “biological sample” refers to a quantity of a substance (a sample) that is isolated from a living thing or formerly living thing. Such substances include, but are not limited to, blood, (e.g., whole blood, dried blood spot), tissue, plasma, serum, sputum, pleural effusion, nasal lavage fluid, Bronchoalveolar Lavage (BAL) fluid, saliva, urine, bone, interstitial fluid, extracellular fluid, organ, or blood vessel. In one embodiment, the biological sample is a sputum sample.

As used herein, the term “sputum sample” refers to a quantity of a substance (a sample) from expectorated secretions from a patient's respiratory tract. The sputum (or phlegm) typically comprises mucous secretion from the bronchi and bronchioles. It may also contain microorganisms (such as bacteria). According to the present invention the sputum sample may be either spontaneous or induced.

As used herein, the term “subject” refers to a mammal, including humans, non-human primates, and non-primate mammals such as members of the rodent genus (including but not limited to mice and rats) and members of the order Lagomorpha (including but not limited to rabbits). In one embodiment, the subject is a human.

As used herein, the term “responder” refers to a COPD patient who through analysis has been identified as someone who will benefit from treatment with a pharmaceutical product of the present invention. A responder will also have a greater response to and derive greater benefit from treatment than a COPD patient who has been identified as a “non-responder”. In the context of the present invention, a responder is a patient suffering from an exacerbation of COPD who has an increased concentration of sputum IL-1α and TNF-α and is thus identified as suffering from an exacerbation associated with a bacterial infection meaning they will derive greater benefit from treatment with an antibiotic agent of the invention.

As used herein, the term “bacterial infection” refers to an infection associated with bacteria. Bacteria that cause respiratory tract infections include Haemophilus influenzae, Moraxella catarrhalis, Streptococcus pneumoniae, Staphylococcus aureus, Pseudomonas aeruginosa, Klebsiella pneumoniae, Escherichia coli, Chlamydophila pneumoniae, Mycoplasma pneumoniae, Legionella pneumophila, Serratia marcescens, Mycobacterium tuberculosis and Bordetella pertussis. Bacteria that cause respiratory tract infections associated with COPD include, in particular, Haemophilus influenza, Moraxella catarrhalis, Streptococcus pneumoniae, Staphylococcus aureus, Pseudomonas aeruginosa. A bacterial infection may be determined by identifying a bacterial pathogen (e.g. Haemophilus influenzae, Moraxella catarrhalis, Streptococcus pneumoniae, Staphylococcus aureus or Pseudomonas aeruginosa), by culture of a biological sample (e.g. sputum sample) or a total aerobic CFU count greater than or equal to 10⁷ cells. Alternatively, a bacterial infection may be determined using polymerase chain reaction assay for detecting bacterial DNA.

As used herein an “effective amount” refers to at least an amount effective, at dosages and for periods of time necessary, to achieve the desired or indicated effect, including a therapeutic or prophylactic result. An effective amount can be provided in one or more administrations.

As used herein, the term “therapeutically effective amount” is at least the minimum concentration required to effect a measurable improvement of a particular disorder. A therapeutically effective amount herein may vary according to factors such as the disease state, age, sex, and weight of the patient, and the ability of the antibody to elicit a desired response in the individual. A therapeutically effective amount is also one in which any toxic or detrimental effects of the antibody are outweighed by the therapeutically beneficial effects. In relation to the present invention, a therapeutically effective amount may relate to the minimum concentration of an antibiotic agent of the invention required to effect a measurable improvement in a patient suffering from an exacerbation of COPD.

As used herein, the term “treatment of an exacerbation of COPD” means ameliorating, stabilising, reducing or eliminating the increased symptoms that are a feature of an exacerbation in a subject.

As used herein, the term “prevention of an acute exacerbation of COPD” means preventing, reducing the incidence or frequency, or reducing the severity of future exacerbations in a subject.

As used herein, the “≥” symbol means “greater than or equal to”.

### COPD and Exacerbation of COPD

Chronic obstructive pulmonary disease (COPD) is a lung disease characterized by chronic obstruction of lung airflow that interferes with normal breathing and is not fully reversible. A COPD diagnosis is confirmed by a simple test called spirometry, which measures how deeply a person can breathe and how fast air can move into and out of the lungs. Such a diagnosis should be considered in any patient who has symptoms of cough, sputum production, or dyspnea (difficult or laboured breathing), and/or a history of exposure to risk factors for the disease. Where spirometry is unavailable, the diagnosis of COPD should be made using all available tools. Clinical symptoms and signs, such as abnormal shortness of breath and increased forced expiratory time, can be used to help with the diagnosis. A low peak flow is consistent with COPD but may not be specific to COPD because it can be caused by other lung diseases and by poor performance during testing. Chronic cough and sputum production often precede the development of airflow limitation by many years, although not all individuals with cough and sputum production go on to develop COPD.

COPD is characterised by chronic obstruction of lung airflow that interferes with normal breathing, and a decline in pulmonary function. COPD is complicated by acute exacerbations (AECOPD) which are transient and stochastic periods of increased COPD symptoms requiring additional medical treatment and often hospitalisation. An exacerbation of COPD (e.g. an acute exacerbation of COPD (AECOPD)) is an event characterised by a sudden worsening of the patient's respiratory symptoms that is beyond normal day-to-day variations. Typically, an exacerbation of COPD leads to a change in medication. Exacerbations may be triggered by a variety of stimuli including exposure to pathogens, such as bacteria and viruses, inhaled irritants such as smoke from cigarettes, allergens, or pollutants. COPD patients with a documented history of one or more acute exacerbations have an increased risk of subsequent exacerbations, particularly bacterial exacerbations.

A reduction in frequency, duration or severity of acute exacerbation or one or more symptoms of an exacerbation may be measured by clinical observation by a doctor or clinician. A reduction in frequency, duration or severity is determined relative to the frequency, duration or severity of an exacerbation or symptom in the same subject not treated according to the methods of the present invention. Suitable clinical observations by an ordinarily skilled clinician may include objective measures of lung function, as well as the frequency with which medical intervention is required. Subjective self-evaluation by the subject may also be used as a measure, for example, using an FDA-recognized subject reported outcome tool or the Exacerbations from Pulmonary Disease Tool (EXACT-PRO).

### Biomarkers

As disclosed herein, biomarkers such as IL-1α and TNF-α can be used in the detection, diagnosis, treatment, and monitoring of patients with COPD. Such markers may be detected at altered concentrations in biological samples from subjects suffering from and/or being treated for an exacerbation of COPD. The altered expression levels can also be used diagnostically (e.g., altered expression levels of one or more biomarkers can serve as a diagnostic to identify a patient suffering from an exacerbation of COPD caused by a bacterial infection).

The altered expression levels can also be used to monitor the efficacy of a course of treatment for an exacerbation caused by a bacterial infection (e.g., if expression levels of a biomarker do not decrease over the course of treatment, this can indicate an ineffective treatment). Accordingly, disclosed herein are new methods of diagnosing, monitoring the progression of treatment, and/or adjusting the dose of a therapeutic agent for treating an inflammatory disorder by determining the expression levels of one or more biomarkers, such as one or more of IL-1α and TNF-α.

In certain embodiments, the biomarkers measured in any of the methods disclosed herein comprise a combination of the following biomarkers: IL-1α and TNF-α.

TNF-α (“tumor necrosis factor-alpha,” “TNF-a”, or “cachectin”) is a cytokine encoded by the TNF gene. IL-1α (“interleukin 1-alpha”, “IL-1a”, “IL1α”, “IL1a” or “Hematopoietin-1”) is a proinflammatory cytokine and through expression of integrins on leukocytes and endothelial cells, initiates and regulates inflammatory responses.

### Reference Concentration

The concentration of one or more biomarkers in a biological sample may be compared to a reference concentration from a reference sample in any of the above-mentioned techniques. The reference concentration may be, (i) the concentration in a biological sample obtained from a healthy subject (i.e. a subject which does not have COPD), (ii) the concentration in a biological sample from a subject with COPD but who does not have an exacerbation (i.e. stable state), or (iii) the concentration in a biological sample obtained from the same subject at a different timepoint, for example when the subject is not experiencing an exacerbation of COPD. Alternatively, the reference concentration may be a concentration or concentration-range that has been assigned as a “normal” (i.e. “healthy”) concentration or concentration-range. The reference concentration may be measured in pg/ml. In some embodiments, the reference concentration is an average of the concentrations in more than one reference sample. In certain embodiments, the reference may be a sample from a patient with the inflammatory disorder prior to or during treatment (e.g., the same patient that is subsequently given a treatment following measurement, or the patient being monitored during treatment).

In respect of the present invention, the reference concentration of IL-1α may be less than 30 pg/ml and the reference concentration of TNF-α may be less than 25 pg/ml wherein said reference concentrations are as measured in a biological sample from a subject with COPD but who does not have an exacerbation (i.e. stable state).

In certain embodiments, the biomarker level from a patient sample is compared to the level in a reference sample. The reference sample may be any of the biological sample types mentioned above. It may be the same sample type collected from the subject. In certain embodiments, the reference level is an average of the level in 2, 3, 4, 5, 6, 7, 8, 9, 10, or more reference samples.

### Antibiotics

Mild to moderate exacerbations of COPD are usually treated with older broad-spectrum antibiotics such as doxycycline, trimethoprim-sulfamethoxazole and amoxicillin-clavulanate potassium (Hunter and King, Am Fam Physician. 2001 Aug. 15; 64(4):603-613). According to NICE (October 2019) antibiotics recommended for adults aged 18 years and over for treating acute exacerbations of COPD are: Amoxicillin, Doxycycline, Clarithromycin, Co-amoxiclav, Co-trimoxazole, Levofloxacin, Piperacillin with tazobactam.

The present invention is directed to an antibiotic agent for use in the treatment of a subgroup of subjects presenting with an exacerbation of COPD. The subgroup of subjects of interest for the present invention are those having an increased concentration of IL-1α and TNF-α in a biological sample (e.g. sputum sample) taken from the subject since these subjects may show an improved clinical response to treatment with an antibiotic of the invention, compared to subjects who do not have an increased concentration of IL-1α and TNF-α in a biological sample (e.g. sputum sample).

The present invention thus provides an antibiotic agent for use in the treatment of an exacerbation of chronic obstructive pulmonary disease (COPD) in a subject, wherein the subject has an increased concentration of IL-1α and TNF-α in a biological sample taken from the subject.

In an embodiment, the increased concentration of IL-1α and TNF-α is indicative that the subject is suffering from an exacerbation of COPD that is associated with a bacterial infection. As such, in an embodiment there is provided an antibiotic agent for use in the treatment of an exacerbation of chronic obstructive pulmonary disease (COPD) in a subject, wherein the subject has an increased concentration of IL-1α and TNF-α in a biological sample taken from the subject wherein the increased concentration of IL-1α and TNF-α is indicative that the subject is suffering from an exacerbation of COPD that is associated with a bacterial infection.

Also provided is an antibiotic agent for use in a method of treating an exacerbation of COPD in a subject, wherein the method comprises: a) measuring the concentration of IL-1α and TNF-α in a biological sample taken from the subject, b) differentiating that the subject is suffering from an exacerbation of COPD that is associated with a bacterial infection if the concentration of IL-1α and TNF-α are increased and, c) administering the antibiotic agent to the subject.

The antibiotic agent of the invention may be administered to a subject through any reasonable route of administration. For example, in an embodiment, the antibiotic agent of the invention is administered orally. In an embodiment, the antibiotic agent of the invention is administered intravenously or via injection.

In an aspect, the present invention is directed to an antibiotic agent for use in a method of treating an exacerbation of COPD in a subject classified as a responder using a method comprising: a) measuring the concentration of IL-1α and TNF-α in a biological sample taken from the subject, b) determining that the subject is a responder if the concentration of IL-1α and TNF-α are increased and, c) administering the antibiotic agent to the subject identified as a responder.

In an embodiment, the bacterial infection is an infection that comprises at least one of Haemophilus influenzae, Streptococcus pneumoniae, Staphylococcus aureus, Moraxella catarrhalis and/or Pseudomonas aeruginosa. In an embodiment, the bacterial infection is an infection that comprises at least Haemophilus influenzae and/or Moraxella catarrhalis. In an embodiment, the bacterial infection is identified using a PCR assay or by performing a microbiological culture.

In an embodiment, the antibiotic agent is of the beta-lactam, macrolide, quinolone or tetracycline class of antibiotic. Table 2 below summarises some exemplar antibiotic agents that have been used in subjects presenting with an exacerbation of COPD. Any antibiotic agent (or any medically compatible combination of antibiotics agents) presented in table 2 below are contemplated within the scope of the present invention (see, HUNTER and KING, Am Fam Physician. 2001 Aug. 15; 64(4): 603-613).

In an embodiment, the antibiotic agent is doxycycline, trimethoprim-sulfamethoxazole, amoxicillin-clavulanate potassium, clarithromycin, azithromycin, levofloxacin, gatifloxacin, moxifloxacin, penicillin, ampicillin, amoxicillin, cefaclor, cefuroxime, cefdinir, cefixime, ceftriaxone, cefotaxime, ceftazidime, cefepime, piperacillin-tazobactam, ticarcillin-clavulanate potassium, levofloxacin/moxifloxacin, gatifloxacin and/or tobramycin.

TABLE 2

| Severity of COPD | | |
|---|---|---|
| exacerbation | Class | Agent |
| Mild to moderate | Tetracycline | Doxycycline |
| exacerbations | First-line | Trimethoprim-sulfamethoxazole (Co-Trimoxazole) |
| | | Amoxicillin-clavulanate potassium (Augmentin)/(Co- |
| | | amoxiclav) |
| | Macrolides | Clarithromycin |
| | | Azithromycin |
| | Fluoroquinolones | Levofloxacin |
| | | Gatifloxacin |
| | | Moxifloxacin |
| | Beta-lactams | Penicillin, ampicillin, amoxicillin |
| | Cephalosporins | Cefaclor, cefuroxime, cefdinir, cefixime |
| Moderate to severe | Cephalosporins | Ceftriaxone |
| exacerbations | | Cefotaxime |
| | | Ceftazidime |
| | | Cefepime |
| | Antipseudomonal | Piperacillin-tazobactam |
| | penicillins | Ticarcillin-clavulanate potassium |
| | Fluoroquinolones | Levofloxacin/moxifloxacin |
| | | Gatifloxacin, |
| | Aminoglycoside | Tobramycin |

A biological sample may be obtained, directly or indirectly, from any cell, tissue, organ, or fluid in a subject. Techniques or methods for obtaining samples from a subject are well known in the art and include, for example, obtaining samples by a mouth swab or a mouth wash, drawing blood, obtaining a biopsy, or obtaining sputum samples. Sputum samples can either be produced spontaneously or may be induced. If an induced sputum sample is used, the subject generally inhales nebulised hypertonic saline (e.g. 3%-7% and may be warmed), which liquefies airway secretions, promotes coughing and allows expectoration of respiratory secretions (which may be collected for example on petri dishes/plates). Isolating components of fluid or tissue samples (e.g., cells or RNA or DNA) may be accomplished using a variety of techniques known to the skilled person. After the sample is obtained, it may be further processed.

In an embodiment, the biological sample is a sputum sample (for example a spontaneous or an induced sputum sample).

As such, in an embodiment there is provided an antibiotic agent for use in the treatment of an exacerbation of chronic obstructive pulmonary disease (COPD) in a subject, wherein the subject has an increased concentration of IL-1α and TNF-α in a biological sample taken from the subject, wherein the biological sample is a sputum sample. Likewise, in an embodiment there is provided an antibiotic agent for use in a method of treating an exacerbation of COPD in a subject, wherein the method comprises: a) measuring the concentration of IL-1α and TNF-α in a biological sample taken from the subject, b) differentiating that the subject is suffering from an exacerbation of COPD that is associated with a bacterial infection if the concentration of IL-1α and TNF-α are increased and, c) administering the antibiotic agent to the subject, wherein the biological sample is a sputum sample.

In an embodiment, the antibiotic agent of the invention or the antibiotic agent for use in a method of treating an exacerbation of COPD is used to treat a subject suffering from an exacerbation of COPD that is associated with a bacterial infection rather than an exacerbation that is associated with a viral or eosinophilic infection, or a pauci exacerbation. In an embodiment, an exacerbation that is associated with a bacterial infection is an exacerbation that is caused by a bacterial infection.

An exacerbation that is “associated with a bacterial infection” means that the subject suffering from an exacerbation has an infection associated with bacteria. An exacerbation that is “associated with a bacterial infection” means that a sputum sample taken from the subject is subjected to a polymerase chain reaction (PCR) test for the presence of bacterial DNA and tests PCR positive for at least one of Haemophilus influenzae, Streptococcus pneumoniae, Streptococcus pyogenes, Staphylococcus aureus, Moraxella catarrhalis and/or Pseudomonas aeruginosa in sputum samples. In an embodiment, the sputum sample tests positive for at least Haemophilus influenzae and/or Moraxella catarrhalis.

An exacerbation that is “associated with a viral infection” is an exacerbation wherein DNA extracted from a sputum of the subject is subjected to a PCR test for the presence of at least one virus and tests positive for at least one virus. An exacerbation that is “associated with an eosinophilic infection” is an exacerbation wherein eosinophils are greater than 3% of non-squamous cells in sputum taken from the subject.

An exacerbation that is labelled a “pauci exacerbation” is an exacerbation that is not bacterial, not viral and not eosinophilic (taking into account the above tests for bacterial/viral/eosinophilic exacerbation).

In an embodiment, the concentration of IL-1α and TNF-α are increased when the concentration of IL-1α is ≥46 pg/mL and the concentration of TNF-α is ≥40 pg/mL in the biological sample taken from the subject. In an embodiment, the concentration of IL-1α and TNF-α are increased when the subject has a sputum IL-1α concentration of ≥46 pg/mL and a sputum TNF-α concentration of >40 pg/mL.

As such, the present invention provides an antibiotic agent for use in the treatment of an exacerbation of chronic obstructive pulmonary disease (COPD) in a subject, wherein the subject has an increased concentration of IL-1α and TNF-α in a biological sample taken from the subject, wherein the biological sample is a sputum sample and wherein the concentration of IL-1α and TNF-α are increased when the concentration of IL-1α is ≥46 pg/mL and the concentration of TNF-α is ≥40 pg/mL in the biological sample. In an embodiment the invention provides an antibiotic agent for use in the treatment of an exacerbation of chronic obstructive pulmonary disease (COPD) in a subject, wherein the subject has an increased concentration of IL-1α and TNF-α in a biological sample taken from the subject, wherein the biological sample is a sputum sample and wherein the concentration of IL-1α and TNF-α are increased when the subject has a sputum IL-1α concentration of ≥46 pg/mL and a sputum TNF-α concentration of ≥40 pg/mL

In an embodiment the present invention provides an antibiotic agent for use in a method of treating an exacerbation of COPD in a subject, wherein the method comprises: a) measuring the concentration of IL-1α and TNF-α in a biological sample taken from the subject, b) differentiating that the subject is suffering from an exacerbation of COPD that is associated with a bacterial infection if the concentration of IL-1α and TNF-α are increased and, c) administering the antibiotic agent to the subject, wherein the biological sample is a sputum sample and wherein the concentration of IL-1α and TNF-α are increased when the concentration of IL-1α is ≥46 pg/mL and the concentration of TNF-α is ≥40 pg/mL in the biological sample. In an embodiment, the present invention provides, an antibiotic agent for use in a method of treating an exacerbation of COPD in a subject, wherein the method comprises: a) measuring the concentration of IL-1α and TNF-α in a biological sample taken from the subject, b) differentiating that the subject is suffering from an exacerbation of COPD that is associated with a bacterial infection if the concentration of IL-1α and TNF-α are increased and, c) administering the antibiotic agent to the subject, wherein the biological sample is a sputum sample and wherein the concentration of IL-1α and TNF-α are increased when the subject has a sputum IL-1α concentration of ≥46 pg/mL and a sputum TNF-α concentration of ≥40 pg/mL.

In an embodiment, the concentration of IL-1α and TNF-α are increased when the concentration of IL-1α is 46 to 50 pg/mL, 50-75 pg/mL, 75-100 pg/mL or greater than 100 pg/mL and the concentration of TNF-α is 40 to 50 pg/mL, 50-75 pg/mL, 75-100 pg/mL or greater than 100 pg/mL in the biological sample taken from the subject. In an embodiment, the concentration of IL-1α and TNF-α are increased when the concentration of IL-1α is ≥46 pg/mL, greater than 50 pg/mL, greater than 75 pg/mL, greater than 100 pg/mL or greater than 150 pg/mL and the concentration of TNF-α is ≥40 pg/mL, greater than 50 pg/mL, greater than 75 pg/mL, greater than 100 pg/mL or greater than 150 pg/mL in the biological sample taken from the subject.

In an embodiment, the concentration of IL-1α and TNF-α are increased when the concentration of IL-1α is 46-75 pg/mL and the concentration of TNF-α is 40-75 pg/mL in the biological sample taken from the subject. In an embodiment, the concentration of IL-1α and TNF-α are increased when the concentration of IL-1α is 50-75 pg/mL and the concentration of TNF-α is 50-75 pg/mL in the biological sample taken from the subject. In an embodiment, the concentration of IL-1α and TNF-α are increased when the concentration of IL-1α is 75-100 pg/mL and the concentration of TNF-α is 75-100 pg/mL in the biological sample taken from the subject.

In an embodiment the concentration of IL-1α and TNF-α are increased when the concentration of IL-1α is ≥46 pg/mL and the concentration of TNF-α is ≥40 pg/mL in the biological sample taken from the subject. In an embodiment the concentration of IL-1α and TNF-α are increased when the concentration of IL-1α is greater than 50 pg/mL and the concentration of TNF-α is greater than 50 pg/mL in the biological sample taken from the subject. In an embodiment the concentration of IL-1α and TNF-α are increased when the concentration of IL-1α is greater than 75 pg/mL and the concentration of TNF-α is greater than 75 pg/mL in the biological sample taken from the subject.

In an embodiment, the concentration of IL-1α and TNF-α are increased when the subject has a sputum IL-1α concentration of 46 to 50 pg/mL, 50-75 pg/mL, 75-100 pg/mL or greater than 100 pg/mL and a sputum TNF-α concentration of 40 to 50 pg/mL, 50-75 pg/mL, 75-100 pg/mL or greater than 100 pg/mL. In an embodiment, the concentration of IL-1α and TNF-α are increased when the subject has a sputum IL-1α concentration of ≥46 pg/mL, greater than 50 pg/mL, greater than 75 pg/mL, greater than 100 pg/mL or greater than 150 pg/mL and the subject has a sputum TNF-α concentration of ≥40 pg/mL, greater than 50 pg/mL, greater than 75 pg/mL, greater than 100 pg/mL or greater than 150 pg/mL.

In an embodiment, the concentration of IL-1α and TNF-α are increased when the subject has a sputum IL-1α concentration of 46-75 pg/mL and a sputum TNF-α concentration of 40-75 pg/mL. In an embodiment, the concentration of IL-1α and TNF-α are increased when the subject has a sputum IL-1α concentration of 50-75 pg/mL and a sputum TNF-α concentration of 50-75 pg/mL. In an embodiment, the concentration of IL-1α and TNF-α are increased when the subject has a sputum IL-1α concentration of 75-100 pg/mL and a sputum TNF-α concentration of 75-100 pg/mL

In an embodiment the concentration of IL-1α and TNF-α are increased when the subject has a sputum IL-1α concentration of ≥46 pg/mL and a sputum TNF-α concentration of ≥40 pg/mL. In an embodiment the concentration of IL-1α and TNF-α are increased when the subject has a sputum IL-1α concentration of greater than 50 pg/mL and a sputum TNF-α concentration of greater than 50 pg/mL. In an embodiment the concentration of IL-1α and TNF-α are increased when the subject has a sputum IL-1α concentration of greater than 75 pg/mL and a sputum TNF-α concentration of greater than 75 pg/mL.

In a separate embodiment the concentration of IL-1α and TNF-α are adjudged to be increased when the concentration of IL-1α and TNF-α in a test biological sample (e.g. sputum sample) is higher than the concentration of IL-1α and TNF-α in a reference biological sample (e.g. sputum sample). In an embodiment the concentration of IL-1α and TNF-α a in the test biological sample (e.g. sputum sample) is greater than 2-fold, greater than 3-fold, greater than 4-fold, greater than 5-fold, greater than 10-fold, greater than 25-fold or greater than 50-fold higher than the concentration of IL-1α and TNF-α in the reference biological sample. In this embodiment, the reference biological sample is either (i) a biological sample obtained from a healthy subject such as a subject which does not have COPD or (ii) a biological sample obtained from a subject with COPD but who does not have an exacerbation. In an embodiment, the reference biological sample is a biological sample obtained from a subject who has not been diagnosed with either COPD nor any other respiratory conditions or diseases. In an embodiment, the reference biological sample is a biological sample obtained from a subject with COPD but who does not have an exacerbation (e.g. the subject at a stable state).

A subject with COPD but who does not have an exacerbation (i.e. a subject at the “stable state”) means for example, a subject who is not substantially presenting with symptoms of exacerbation, for example a COPD patient at a routine medical examination. For example, the subject is not substantially presenting with any of the following symptoms: wheezing, persistent cough, hyperventilation, noticeable increase in mucus production, fever, confusion etc. The concentration of IL-1α at a stable state should be less than 30 pg/ml and the concentration of TNF-α at the stable state should be less than 25 pg/mL.

In an embodiment the concentration of IL-1α and TNF-α are increased when the concentration of IL-1α and TNF-α are greater than 1.5-fold, greater than 2-fold, greater than 3-fold, greater than 5-fold or greater than 10-fold compared to the concentration of IL-1α and TNF-α measured at the stable state. In an embodiment the concentration of IL-1α and TNF-α are increased when the concentration of IL-1α and TNF-α are greater than 1.5-fold, greater than 2-fold, greater than 3-fold, greater than 5-fold or greater than 10-fold compared to the concentration of IL-1α and TNF-α measured in a biological sample taken from the subject at the stable state. In an embodiment the concentration of IL-1α and TNF-α are increased when the sputum concentration of IL-1α and TNF-α are greater than 1.5-fold, greater than 2-fold, greater than 3-fold, greater than 5-fold or greater than 10-fold compared to the sputum concentration of IL-1α and TNF-α measured at the stable state.

In an embodiment the subject is an animal, preferably a mammal, including humans, non-human primates and non-primate mammals such as members of the rodent genus (including but not limited to mice and rats), the Cavia genus (including but not limited to guinea pigs) and members of the order Lagomorpha (including but not limited to rabbits). In a preferred embodiment the subject is a human. As used herein, the term “subject” may be used interchangeably with the word “patient” i.e. in an embodiment the subject is a patient.

In an embodiment, the subject may be an adult human, for example, aged between 18 and 40 or between 50 and 70 or between 40 and 85 years of age. The subject has a previous history of Chronic Obstructive Pulmonary Disease (COPD), particularly, a previous history of moderate and severe Acute Exacerbation of Chronic Obstructive Pulmonary Disease (AECOPD). For example, a confirmed diagnosis of COPD, categorised as moderate, severe, or very severe according to the Global Initiative for Chronic Obstructive Lung Disease (GOLD) classification. The Global Strategy for the Diagnosis, Management and Prevention of COPD prepared by GOLD state that COPD should be considered in any patient with dyspnea, chronic cough or sputum production, and/or a history of exposure to risk factors for the disease, such as tobacco smoking, occupation, or pollutants. A spirometry assessment, measuring airflow limitation, is required to establish diagnosis. The classification of airflow limitation severity in COPD outlined in the GOLD strategy is shown in Table 1.

TABLE 1
Classification of airflow limitation severity in COPD (Based
on post-bronchodilator FEV₁) In patients with FEV₁/FVC < 0.70

| | | |
|---|---|---|
| GOLD 1 | Mild | FEV₁ ≥ 80% predicted |
| GOLD 2 | Moderate | 50% ≤ FEV₁ < 80% predicted |
| GOLD 3 | Severe | 30% ≤ FEV₁ < 50% predicted |
| GOLD 4 | Very Severe | FEV₁ < 30% predicted |

COPD assessment also includes analysis of patient symptoms, and this can be performed using comprehensive disease-specific health status questionnaires such as the Chronic Respiratory Questionnaire (CRQ) and St. George's Respiratory Questionnaire (SGRQ). For routine practice the COPD Assessment Test (CAT™) and The COPD Control Questionnaire (The CCQ©) have been developed. The CAT™ and CCQ© tests do not categorise patients for the purpose of treatment, however for the SRGQ assessment a symptom score ≥25 may be used as the threshold for considered regular treatment for breathlessness. The equivalent threshold for the CAT™ is 10. A simple assessment of breathlessness is the Modified British Medical Research Council (mMRC) Questionnaire. According to the GOLD strategy, of the patients classified at the GOLD 2 (moderate) stage, approximately 20% may experience frequent exacerbations requiring antibiotic and/or systemic corticosteroid therapy in addition to regular maintenance therapy. The risk of exacerbations is significantly higher for patients classified as GOLD 3 (severe) and GOLD 4 (very severe). The “ABCD” assessment tool is further used to understand a COPD patient's severity of disease. This assessment combines the patient's spirometry analysis with their exacerbation history and symptom assessment to give a spirometric grade combined with an “ABCD” group. In some embodiments, the subject has GOLD 2 (moderate), GOLD 3 (severe) or GOLD 4 (very severe) COPD status. The subject may be one that has experienced at least one (e.g. 2 or more, 3 or more) episodes of acute exacerbation in chronic obstructive pulmonary disease (AECOPD), particularly at least one (e.g. 2 or more, 3 or more) episodes of acute exacerbation in chronic obstructive pulmonary disease (AECOPD) within a period of 12 months. Yet more particularly the subject has experienced at least one (e.g. 2 or more, 3 or more) episode of acute exacerbation in chronic obstructive pulmonary disease (AECOPD) in the preceding 12 months. The subject may be a subject having bronchiectasis.

The level of expression of a biomarker in a sample obtained from a subject may be assayed by any of a wide variety of techniques and methods. In some embodiments, measuring and determining the biomarker level in a sample can comprise a technique from one or more of the following: fluorescence-activated cell sorting (FACS), polymerase chain reaction (PCR), quantitative or real-time PCR (qPCR), gel electrophoresis, high-throughput sequencing, next generation sequencing, mass spectrometry, RNA sequencing, enzyme-linked immunosorbent spot (ELISpot), enzyme-linked immunosorbent assay (ELISA), microarray assay, quantitative biphasic calcium phosphate (BCP), Northern blot assay, Southern blot assay, Western blot assay, immunohistochemical assay, binding assay and combinations thereof. In an embodiment, the method is Northern blot, ELISA, FACS, or flow cytometry.

The invention provides a binding immunoassay. The invention can use any ELISA format, including those conventionally known as direct ELISA, indirect ELISA, sandwich ELISA, and competitive ELISA. In an embodiment the assay is a sandwich ELISA.

In an embodiment, the concentration of IL-1α and TNF-α are measured by an immunoassay. In an embodiment, the concentration of IL-1α and TNF-α are measured by an immunoassay wherein the immunoassay is an ELISA based assay. In an embodiment, the concentration of IL-1α and TNF-α are measured by a sandwich ELISA assay. In an embodiment, the concentration of IL-1α and TNF-α are measured by an immunoassay, wherein the immunoassay is a Meso Scale Discovery (MSD) assay. The MSD electrochemiluminescence ELISA based assay may be performed essentially as described in [Dabitao et al 2011 (J Immunol Methods. 2011; 372(1-2): 71-77)] and [Burguillos M A (2013) Methods and Protocols, Methods in Molecular Biology, vol. 1041, DOI 10.1007/978-1-62703-520-0_11].

The Meso-Scale Discovery (MSD) immunoassay platforms provide an electrochemiluminescence detection system that is capable of detecting low levels of cytokines. The MSD ELISA based assay enables the measurement of a panel of cytokines from a single sample. MSD provides a plate coated with anti-species antibody (capture antibody) that allows the immobilization of the cytokines of the invention in the sample. Later on, the detecting antibody is added together with a SULFO-TAG™ label. The user adds an MSD Read Buffer (with the appropriate chemical environment for the electrochemiluminescence reaction plus co-reactants that enhance the signal) and quantifies the emitted light.

In an embodiment, the concentration of IL-1α and TNF-α are measured in a single biological sample taken from the subject. In an embodiment, the concentration of IL-1α and TNF-α are measured in multiple biological samples taken from the subject and an average concentration obtained. In an embodiment, the biological samples are diluted prior to analysis.

In an embodiment, the concentration of IL-1α and TNF-α are measured in multiple biological samples taken from the subject and the concentration of IL-1α and TNF-α are compared over time. For example, a subject presenting with an exacerbation of COPD may have a biological sample taken from them for measurement of IL-1α and TNF-α when they initially present with symptoms of an exacerbation. The subject may then have a follow-up biological samples taken from them after they have received, for example, the antibiotic agent of the invention, to measure the concentration of IL-1α and TNF-α (i.e. for comparison against the concentrations of IL-1α and TNF-α upon initial presentation).

Exacerbations of COPD may be treated with systemic corticosteroids and/or antibiotics. The present invention enables clinicians to determine whether or not a subject is presenting with an exacerbation of COPD that is caused by a bacterial infection (i.e. if the concentration of IL-1α and TNF-α are increased in a biological sample taken from that subject). If a subject is then identified as suffering from an exacerbation of COPD that is associated with a bacterial infection (i.e. if the concentration of IL-1α and TNF-α are increased in a biological sample taken from the subject), an antibiotic agent may be preferred to, or used in combination with, a corticosteroid based therapy.

Therefore, in an embodiment the subject is not administered systemic corticosteroid-based therapy. The subject may however continue their use of a maintenance inhaled corticosteroid. In an embodiment, the antibiotic agent of the invention is used in preference to a systemic corticosteroid-based therapy (e.g. use of a systemic corticosteroid). In an embodiment the subject is administered a systemic corticosteroid in combination with the antibiotic agent.

The antibiotics of the present invention may in a further aspect be used in the treatment of a disease, disorder, manifestation or exacerbation that is associated with a bacterial infection in a subject, wherein the subject is identified as suffering from a disease, disorder, manifestation or exacerbation that is associated with a bacterial infection when said subject has an increased concentration of IL-1α and TNF-α in a biological sample taken from the subject.

As used herein disease and/or disorder refers to conditions that have a negative impact on the health of a subject and characterised by specific signs and symptoms. Most notably the diseases or disorders as referred to in respect of the present invention may for example be inflammatory diseases or disorders, particularly those caused by bacterial infections. As used herein manifestations means the signs/symptoms of said disease or disorder. As used herein exacerbation means an event characterised by a worsening of signs/symptoms.

In an embodiment, the increased concentration of IL-1α and TNF-α is indicative that the subject is suffering from a disease, disorder, manifestation, or exacerbation which is associated with a bacterial infection as opposed to, for example, a viral infection. As a result of this analysis, a clinician may administer the antibiotic agent with an increased probability that said antibiotic agent will result in successful clinical outcomes e.g. eliminating the infection, alleviating signs/symptoms etc.

Also provided is an antibiotic agent for use in a method of treating a subject, wherein the method comprises: a) measuring the concentration of IL-1α and TNF-α in a biological sample taken from the subject, b) differentiating that the subject is suffering from a disease, disorder, manifestation or exacerbation that is associated with a bacterial infection if the concentration of IL-1α and TNF-α are increased and, c) administering the antibiotic agent to the subject.

The antibiotic agent of the invention may be administered to a subject through any reasonable route of administration. For example, in an embodiment, the antibiotic agent of the invention is administered orally. In an embodiment, the antibiotic agent of the invention is administered intravenously or via injection.

In an embodiment, the bacterial infection is an infection that comprises at least one of Haemophilus influenzae, Streptococcus pneumoniae, Staphylococcus aureus, Moraxella catarrhalis and/or Pseudomonas aeruginosa. In an embodiment, the bacterial infection is an infection that comprises at least Haemophilus influenzae and/or Moraxella catarrhalis. In an embodiment, the bacterial infection is identified or confirmed using a PCR assay or by performing a microbiological culture.

### Method of Treatment

The method of the invention comprises providing a course of treatment based on the prognosis and/or diagnosis. In this regard, a patient is first diagnosed as suffering from an exacerbation of COPD that is associated with at least one or more bacterial infections. Once this has been identified, the patient is treated with an antibiotic agent.

The invention thus provides a method of treating an exacerbation of COPD in a subject comprising the steps of:
a) measuring the concentration of IL-1α and TNF-α in a biological sample taken from the subject,
b) differentiating that the subject is suffering from an exacerbation of COPD that is associated with a bacterial infection if the concentration of IL-1α and TNF-α are increased and,
c) administering a therapeutically effective amount of an antibiotic agent to the subject.

In an embodiment step a) comprises measurement of a panel of up to 10, up to 20, up to 30 or up to 50 cytokines wherein the panel comprises IL-1α and TNF-α.

In an embodiment, the antibiotic agent that is administered to the subject (at a therapeutically effective amount) is of the beta-lactam, macrolide, quinolone or tetracycline class of antibiotic. In an embodiment, the antibiotic agent is doxycycline, trimethoprim-sulfamethoxazole, amoxicillin-clavulanate potassium, clarithromycin, azithromycin, levofloxacin, gatifloxacin, moxifloxacin, penicillin, ampicillin, amoxicillin, cefaclor, cefuroxime, cefdinir, cefixime, ceftriaxone, cefotaxime, ceftazidime, cefepime, piperacillin-tazobactam, ticarcillin-clavulanate potassium, levofloxacin/moxifloxacin, gatifloxacin and/or tobramycin.

In an embodiment the biological sample is a sputum sample. As such, in an embodiment, there is provided a method of treating an exacerbation of COPD in a subject comprising the steps of: a) measuring the concentration of IL-1α and TNF-α in a biological sample taken from the subject, b) differentiating that the subject is suffering from an exacerbation of COPD that is associated with a bacterial infection if the concentration of IL-1α and TNF-α are increased and, c) administering a therapeutically effective amount of an antibiotic agent to the subject wherein the biological sample is a sputum sample.

Step b) of the method of treatment of the invention, comprises differentiating that the subject is suffering from an exacerbation of COPD that is associated with a bacterial infection if the concentration of IL-1α and TNF-α are increased. In an embodiment the subject is suffering from an exacerbation of COPD that is associated with a bacterial infection, rather than an exacerbation that is associated with a viral or eosinophilic infection, or a pauci exacerbation.

In an embodiment, the concentration of IL-1α and TNF-α are increased when the concentration of IL-1α is ≥46 pg/mL and the concentration of TNF-α is ≥40 pg/mL in the biological sample taken from the subject. In an embodiment, the concentration of IL-1α and TNF-α are increased when the subject has a sputum IL-1α concentration of ≥46 pg/mL and a sputum TNF-α concentration of ≥40 pg/mL.

As such, in an embodiment, there is provided a method of treating an exacerbation of COPD in a subject comprising the steps of: a) measuring the concentration of IL-1α and TNF-α in a biological sample taken from the subject, b) differentiating that the subject is suffering from an exacerbation of COPD that is associated with a bacterial infection if the concentration of IL-1α and TNF-α are increased and, c) administering a therapeutically effective amount of an antibiotic agent to the subject wherein the biological sample is a sputum sample and wherein the concentration of IL-1α and TNF-α are increased when the concentration of IL-1α is ≥46 pg/mL and the concentration of TNF-α is ≥40 pg/mL in the biological sample. In an embodiment there is provided a method of treating an exacerbation of COPD in a subject comprising the steps of: a) measuring the concentration of IL-1α and TNF-α in a biological sample taken from the subject, b) differentiating that the subject is suffering from an exacerbation of COPD that is associated with a bacterial infection if the concentration of IL-1α and TNF-α are increased and, c) administering a therapeutically effective amount of an antibiotic agent to the subject wherein the biological sample is a sputum sample and wherein the concentration of IL-1α and TNF-α are increased when the subject has a sputum IL-1α concentration of ≥46 pg/mL and a sputum TNF-α concentration of ≥40 pg/mL.

In a separate embodiment the concentration of IL-1α and TNF-α are adjudged to be increased when the concentration of IL-1α and TNF-α in a test biological sample (e.g. sputum sample) is higher than the concentration of IL-1α and TNF-α in a reference biological sample (e.g. sputum sample). In an embodiment the concentration of IL-1α and TNF-α a in the test biological sample (e.g. sputum sample) is greater than 2-fold, greater than 3-fold, greater than 4-fold, greater than 5-fold, greater than 10-fold, greater than 25-fold or greater than 50-fold higher than the concentration of IL-1α and TNF-α in the reference biological sample. In this embodiment, the reference biological sample is either (i) a biological sample obtained from a healthy subject such as a subject which does not have COPD or (ii) a biological sample obtained from a subject with COPD but who does not have an exacerbation. In an embodiment, the reference biological sample is a biological sample obtained from a subject who has not been diagnosed with either COPD nor any other respiratory conditions or diseases. In an embodiment, the reference biological sample is a biological sample obtained from a subject with COPD but who does not have an exacerbation (e.g. the subject at a stable state).

In an embodiment, the method of treatment of the invention is a method of treating a human. As such, in an embodiment, the subject is a human.

In an embodiment, step a) comprises measurement of cytokines using an immunoassay, for example an ELISA-based immunoassay (e.g. an MSD multiplex assay).

In an embodiment the bacterial infection is an infection that comprises at least one of Haemophilus influenzae, Streptococcus pneumoniae, Staphylococcus aureus, Moraxella catarrhalis and/or Pseudomonas aeruginosa.

In an embodiment the subject is not administered systemic corticosteroid-based therapy. In an embodiment the antibiotic agent that is administered to the subject (at a therapeutically effective amount) in step c) of the method of treatment of the invention is administered in preference to a systemic corticosteroid(s). In an embodiment, step c) comprises administering a therapeutically effective amount of an antibiotic agent to the subject in combination with a systemic corticosteroid(s).

In a further aspect, the invention provides a method of treating disease, disorder, manifestation, or exacerbation that is associated with a bacterial infection in a subject comprising the steps of:
a) measuring the concentration of IL-1α and TNF-α in a biological sample taken from the subject,
b) differentiating that the subject is suffering from a disease, disorder, manifestation, or exacerbation that is associated with a bacterial infection if the concentration of IL-1α and TNF-α are increased and,
c) administering a therapeutically effective amount of an antibiotic agent to the subject.

### Method of Diagnosis

The present invention can be used either to diagnose and then treat exacerbations of COPD or alternatively just to diagnose what subtype of exacerbation a patient is experiencing. The method of diagnosis of the invention provides a rapid and accurate test that can be used to differentiate patients suffering from an exacerbation of their COPD that is caused by bacterial infection(s) from patients suffering from an exacerbation that is caused by other factors (e.g. viral).

In a further aspect, the present invention thus provides a method for diagnosing that a subject is suffering from an exacerbation of COPD that is associated with a bacterial infection the method comprising:
a) measuring the concentration of IL-1α and TNF-α in a biological sample taken from the subject, and
b) detecting that the concentration of IL-1α and TNF-α are increased.

In a further aspect, the present invention provides a method for diagnosing that a subject is suffering from a disease, disorder, manifestation, or exacerbation that is associated with a bacterial infection the method comprising:
a) measuring the concentration of IL-1α and TNF-α in a biological sample taken from the subject, and
b) detecting that the concentration of IL-1α and TNF-α are increased.

In an embodiment, the method of diagnosis of the invention is performed ex vivo.

In an embodiment, step a) of the method of diagnosis of the invention, comprises measurement of a panel of up to 10, up to 20, up to 30 or up to 50 cytokines wherein the panel comprises IL-1α and TNF-α.

In an embodiment, the biological sample taken from the subject in step a) of the method of diagnosis of the invention, is a sputum sample. Thus, there is provided a method for diagnosing that a subject is suffering from an exacerbation of COPD that is associated with a bacterial infection the method comprising: a) measuring the concentration of IL-1α and TNF-α in a biological sample taken from the subject, and b) detecting that the concentration of IL-1α and TNF-α are increased, wherein the biological sample is a sputum sample.

In an embodiment, the method of diagnosis of the invention is a method of diagnosing that the subject is suffering from an exacerbation of COPD that is associated with a bacterial infection rather than an exacerbation that is associated with a viral or eosinophilic infection, or a pauci exacerbation.

Step b) of the method of diagnosis of the invention comprises detecting that the concentration of IL-1α and TNF-α are increased. In an embodiment, the concentration of IL-1α and TNF-α are increased when the concentration of IL-1α is ≥46 pg/mL and the concentration of TNF-α is ≥40 pg/mL in the biological sample taken from the subject. In an embodiment, the concentration of IL-1α and TNF-α are increased when the subject has a sputum IL-1α concentration of ≥46 pg/mL and a sputum TNF-α concentration of ≥40 pg/mL. As such, there is provided, a method for diagnosing that a subject is suffering from an exacerbation of COPD that is associated with a bacterial infection the method comprising: a) measuring the concentration of IL-1α and TNF-α in a biological sample taken from the subject, and b) detecting that the concentration of IL-1α and TNF-α are increased, wherein the biological sample is a sputum sample and wherein the concentration of IL-1α and TNF-α are increased when the subject has a sputum IL-1α concentration of ≥46 pg/mL and a sputum TNF-α concentration of ≥40 pg/mL.

In a separate embodiment the concentration of IL-1α and TNF-α are adjudged to be increased when the concentration of IL-1α and TNF-α in a test biological sample (e.g. sputum sample) is higher than the concentration of IL-1α and TNF-α in a reference biological sample (e.g. sputum sample). In an embodiment the concentration of IL-1α and TNF-α a in the test biological sample (e.g. sputum sample) is greater than 2-fold, greater than 3-fold, greater than 4-fold, greater than 5-fold, greater than 10-fold, greater than 25-fold or greater than 50-fold higher than the concentration of IL-1α and TNF-α in the reference biological sample. In this embodiment, the reference biological sample is either (i) a biological sample obtained from a healthy subject such as a subject which does not have COPD or (ii) a biological sample obtained from a subject with COPD but who does not have an exacerbation.

In an embodiment, the method of diagnosis of the invention is a method of diagnosing a human subject. Therefore, in an embodiment, the subject is a human.

In an embodiment, the concentration of IL-1α and TNF-α are measured by an immunoassay. In an embodiment, the concentration of IL-1α and TNF-α are measured by an immunoassay wherein the immunoassay is an ELISA based assay. In an embodiment, the concentration of IL-1α and TNF-α are measured by a sandwich ELISA assay. In an embodiment, the concentration of IL-1α and TNF-α are measured by an immunoassay, wherein the immunoassay is a Meso Scale Discovery (MSD) ELISA assay.

There is a number of causes of exacerbations of COPD, only one of which is a bacterial infection. The present invention provides artificial intelligence/machine learning systems, e.g., neural networks, for providing an improved, more accurate determination of the type of exacerbation a subject is suffering from. By providing the neural network system with a sufficiently large training data set the neural network may more accurately predict an individual's likelihood of suffering from an exacerbation that is associated with a bacterial infection. In addition, machine learning systems can evolve over time, as more data becomes available, to make even more accurate predictions. Therefore, in an embodiment, a machine learning model is trained to determine whether (or not) a subject is suffering from an exacerbation of COPD that is associated with a bacterial infection on the basis of sputum cytokine concentrations (for example sputum IL-1α and TNF-α concentration).

In an embodiment the exacerbation of COPD that is caused by a bacterial infection that is diagnosed using the method of diagnosis of the invention, is a bacterial infection that comprises at least one of Haemophilus influenzae, Streptococcus pneumoniae, Streptococcus pyogenes, Staphylococcus aureus, Moraxella catarrhalis and/or Pseudomonas aeruginosa.

### Use in Manufacture

In a further aspect, there is provided the use of an antibiotic agent for the manufacture of a medicament for the treatment of an exacerbation of COPD in a subject, wherein the subject has an increased concentration of IL-1α and TNF-α in a biological sample taken from the subject.

In a yet further aspect, there is provided the use of an antibiotic agent for the manufacture of a medicament for use in a method of treating an exacerbation of COPD in a subject wherein the method comprises measuring the concentration of IL-1α and TNF-α in a biological sample taken from the subject, differentiating that the subject is suffering from an exacerbation of COPD associated with a bacterial infection if the concentration of IL-1α and TNF-α are increased and then administering the antibiotic agent to the subject.

There is further provided the use of an antibiotic agent for the manufacture of a medicament for the treatment of a disease, disorder, manifestation or exacerbation that is associated with a bacterial infection in a subject, wherein the subject is identified as suffering from a disease, disorder, manifestation or exacerbation that is associated with a bacterial infection when said subject has an increased concentration of IL-1α and TNF-α in a biological sample taken from the subject.

There is further provided the use of an antibiotic agent for the manufacture of a medicament for use in a method of treating a disease, disorder, manifestation, or exacerbation that is associated with a bacterial infection wherein the method comprises measuring the concentration of IL-1α and TNF-α in a biological sample taken from the subject in order to identify that said subject is suffering from a disease, disorder, manifestation, or exacerbation that is associated with a bacterial infection (for example as opposed to a viral infection).

### Kits

The invention also provides kits for practicing one or more of the above-described methods that include at least one reagent specific for a COPD biomarker described herein, wherein the COPD biomarkers comprise IL-1α and TNF-α. The expression of the one or more biomarkers can be determined using said reagent that detects the one or more biomarkers.

A kit of the present invention may include reagents that are labelled compounds or agents useful to detect a polypeptide or an mRNA encoding a polypeptide corresponding to a COPD biomarker disclosed herein in a biological sample and means for determining the amount of the polypeptide or mRNA in the sample (e.g., an antibody that binds the polypeptide or an oligonucleotide probe that binds to DNA or mRNA encoding the polypeptide). One type of such reagent suitable for binding with a polypeptide corresponding to a COPD biomarker is an antibody or fragment thereof (including an antibody derivative) that binds to a marker of interest. Additionally, suitable reagents for binding with a nucleic acid (e.g., genomic DNA, mRNA, spliced mRNA, cDNA) include complementary nucleic acids. A variety of different array formats are known in the art, with a wide variety of different probe structures, substrate compositions and attachment technologies. In further embodiments, the reagent is directly or indirectly labelled with a detectable substance.

For antibody-based kits, the kit can comprise, for example: (1) a first antibody (e.g., attached to a solid support) which binds a polypeptide corresponding to a biomarker of the invention; and, optionally (2) a second, different antibody that binds to either the polypeptide or the first antibody and is conjugated to a detectable label.

The kit can also comprise other components, such as a buffering agent, a preservative, a protein stabilizing agent, and/or components necessary for detecting the detectable label. The kit may include reagents employed in the various methods, such as devices for withdrawing and handling blood samples, second stage antibodies, ELISA reagents; tubes, spin columns, and the like. Each component of the kit can be enclosed within an individual container and all of the various containers can be within a single package.

In addition to the above components, the subject kits may further include instructions for practicing the subject methods and for interpreting the results of the assays performed using the kit. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc.

The following examples are intended for illustration only and are not intended to limit the scope of the invention in any way.

### EXAMPLES

### Example 1: Clinical Study

Acute Exacerbation and Respiratory Infections in COPD (AERIS/EPI-HIP-001 BOD UK) was a longitudinal epidemiological study [Wilkinson et al. Thorax 2017; Mayhew et al. Thorax 2018]. The primary objective of the AERIS study was to estimate the incidence of all-cause acute exacerbations (AECOPD) and of AECOPD with sputum containing bacterial pathogens (overall and by species).

Patients with COPD aged 40-85 were followed monthly for 2 years and reviewed within 72 hours of onset of symptoms of AECOPD. Exacerbations were detected using daily electronic diary cards. Blood, sputum, nasopharyngeal and urine samples were collected at prespecified timepoints. Molecular diagnostic and typing techniques were used to describe the dynamics of airway infection during AECOPD and stable disease, and associations with clinical outcome [Bourne S. et al., Acute Exacerbation and Respiratory Infections in COPD (AERIS): protocol for a prospective, observational cohort study. BMJ Open., 4, e004546 (2014)].

In the context of the clinical study AERIS/EPI-HIP-001 BOD UK (ClinicalTrials.gov (NCT01360398)), with particular reference to the tertiary objectives described in the study protocol [Aliquots of sputum samples to perform molecular typing and to describe and compare selected biomarkers in AECOPD (all cause and bacterial) and stable COPD] it was proposed to study the relationships between cytokines expression data in sputum and patients' clinical data in order to better characterize and understand the COPD disease and find new relationships.

### Example 2: Study Design & Experimental Plan

The study design of the clinical study AERIS/EPI-HIP-001 is described in the Clinical Study Protocol (Bourne et al, BMJ Open., 4 e004546 (2014)).

The cytokine and chemokine quantification profiles from sputum samples collected at routine and at exacerbation visits were measured by the Meso Scale Discovery (MSD) assay. Because of patients who were lost to follow-up visits or were withdrew from the study, mainly during the second year, we tested only samples collected during the first year to avoid a possible bias due to missing outcome data. We selected only subjects with at least 3 available visits (with or without exacerbation).

Cytokine and chemokine quantification profiles: For each human sputum PBS sample, the concentration (expressed as pg/ml) of 30 cytokines and chemokines was measured using the V-plex Human Cytokine 30-Plex Panel Kit form MSD.

The Human Cytokine 30-Plex Panel Kit consists of 3 independent 10-plex panels—Proinflammatory Panel 1 (human), Cytokine Panel 1 (human), and Chemokine Panel 1 (human). These panels are composed of assays against human cytokines and chemokines that are involved in process such as inflammation, the Th1/Th2 pathway, chemotaxis, the Th17 pathway, angiogenesis, and immune system regulation.

MSD technology is an electrochemiluminescence detection technology which uses electrochemical stimulation of reporter molecules conjugated to biological components to generate a light signal measured by photodetectors. The instrument measures the intensity of emitted light (which is proportional to the amount of analyte in the sample) and provides a quantitative measure of each cytokines. The V-plex Human Cytokine 30-Plex Panel Kit consists of 3 independent multi-spot 10-plex panels: Cytokine Panel 1 (human), Chemokine Panel 1 (human) and Proinflammatory Panel 1 (human) that detect secreted biomarkers in human fluids. The complete list of cytokines and chemokines quantified is summarized in Table 3.

TABLE 3

| Cytokines | Chemokines | Proinflammatory |
|---|---|---|
| GM-CSF | Eotaxin | IFN-γ |
| IL-1α | MIP-1β | IL-1β |
| IL-5 | Eotaxin-3 | IL-2 |
| IL-7 | TARC | IL-4 |
| IL-12/IL-23p40 | IP-10 | IL-6 |
| IL-15 | MIP-1α | IL-8 |
| IL-16 | IL-8(HA) | IL-10 |
| IL-17A | MCP-1 | IL-12p70 |
| TNF-β | MDC | IL-13 |
| VEGF-A | MCP-4 | TNF-α |

### Example 3: Materials and Methods

Materials: The Human PBS sputum samples were obtained from the AERIS study (ClinicalTrials.gov (NCT01360398)).

Samples were processed essentially according to manufactures recommendations (V-plex Human Cytokine 30-Plex Panel Kit, Meso Scale Discovery). Sputum samples were diluted prior to analysis using a either a 2-fold dilution (cytokine/proinflammatory panel) or 4-fold dilution (chemokines) with the MSD reagent “Diluent 2” or “Diluent 43” respectively.

In brief the procedure was undertaken as follows;
1. Washed and Added sample: Plates were washed with at least 150 μl/well of MSD Wash Buffer. 50 μl of prepared sample, calibrator, or control were added per well. Plate were then sealed with an adhesive plate seal and incubated at room temperature with shaking for 2 hours. The calibration curves were prepared in duplicate.
2. Washed and Added Detection Antibody Solution: Plates were re-washed 3 times with 150 μl/well of MSD wash buffer. Subsequently 25 μl of detection antibody solution was added to each well and the plate sealed (as described above). Plates were incubated at room temperature with shaking for 2 hours.
3. Washed and Added Read Buffer: Plates were re-washed 3 times again with 150 μl/well of MSD wash buffer. 150 μl of 2× Read Buffer was then added to each well and the plate incubated at room temperature for 10 minutes.
4. Read: Plates were then read on the MSD Instrument according to manufacturer's instructions (QuickPlex SQ 120 Reader).

### Example 4: Study Results

Each of the 936 sputum samples listed in the inventory file and collected from 99 patients at routine and exacerbation visits have been assayed by Meso Scale Discovery technology, and 30 cytokines/chemokines have been quantified.

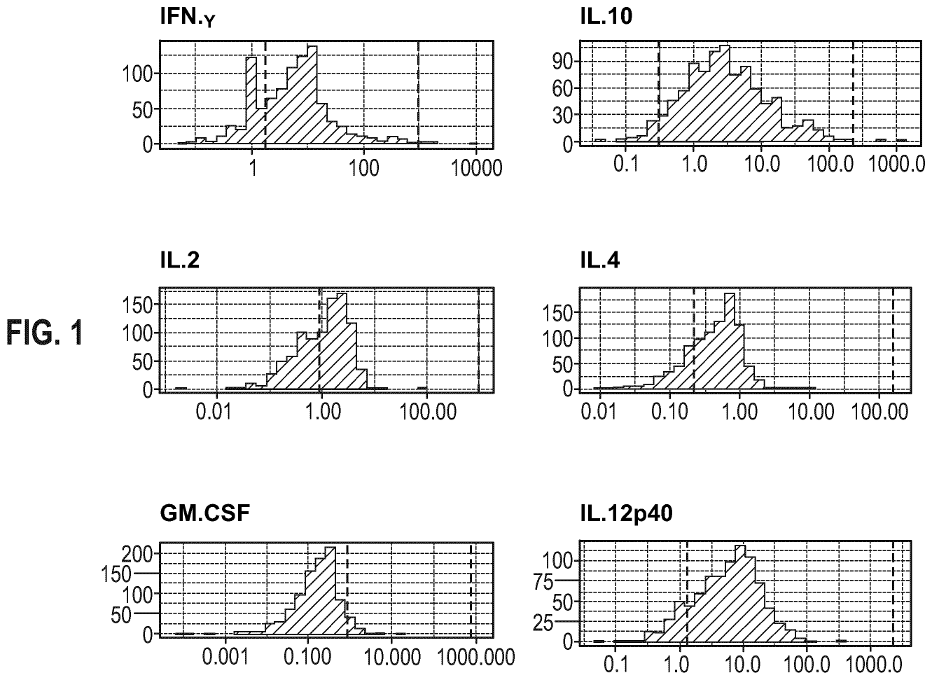

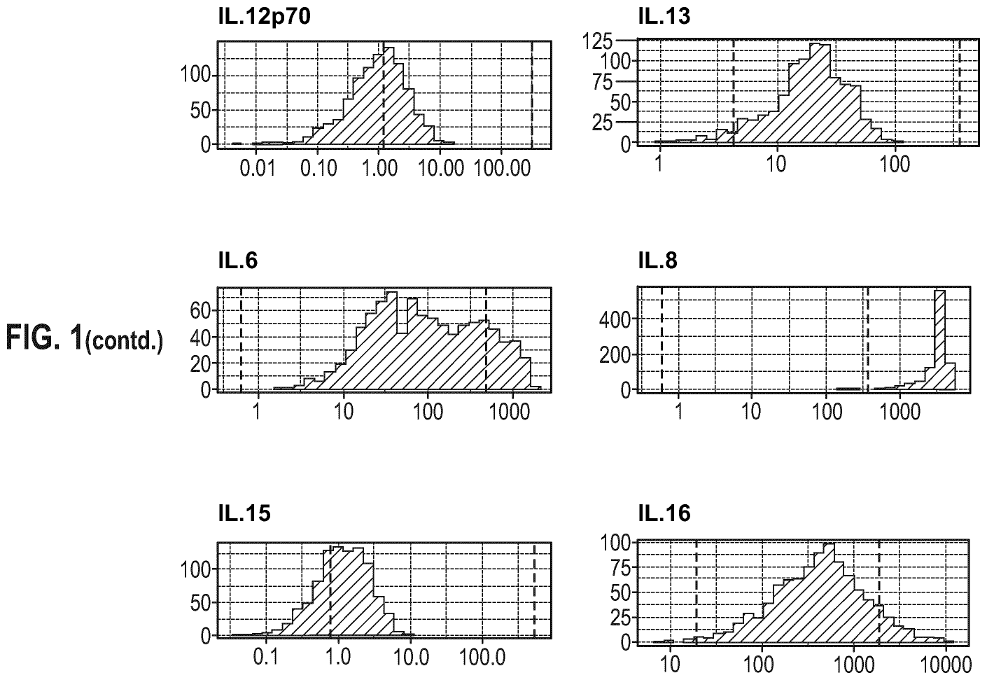

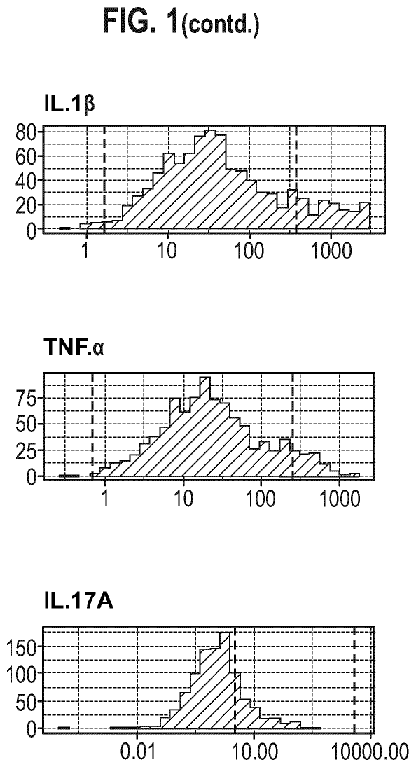

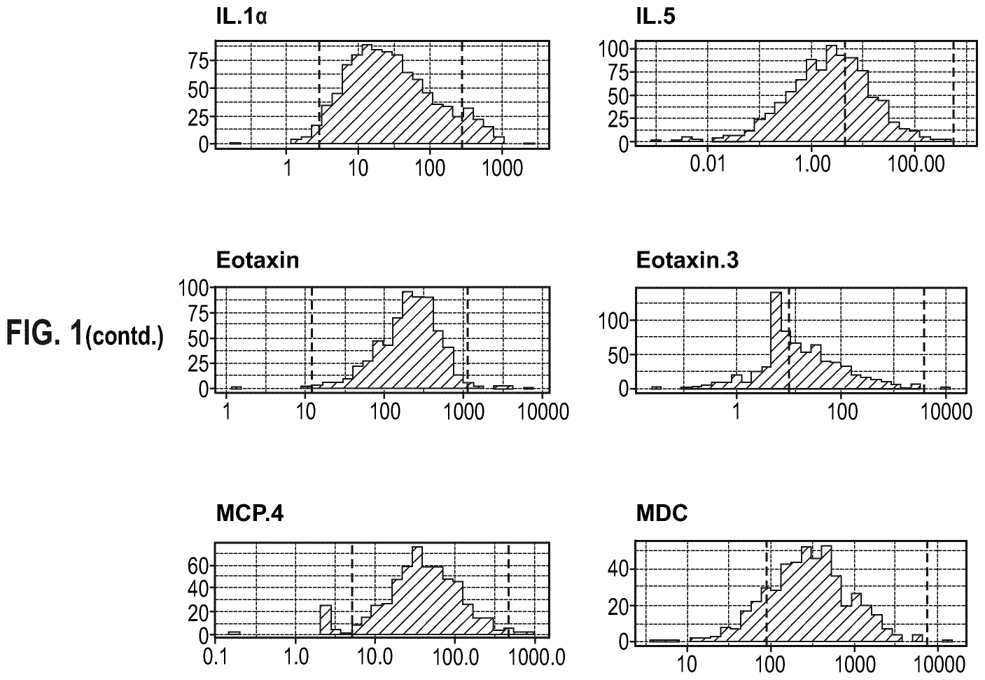

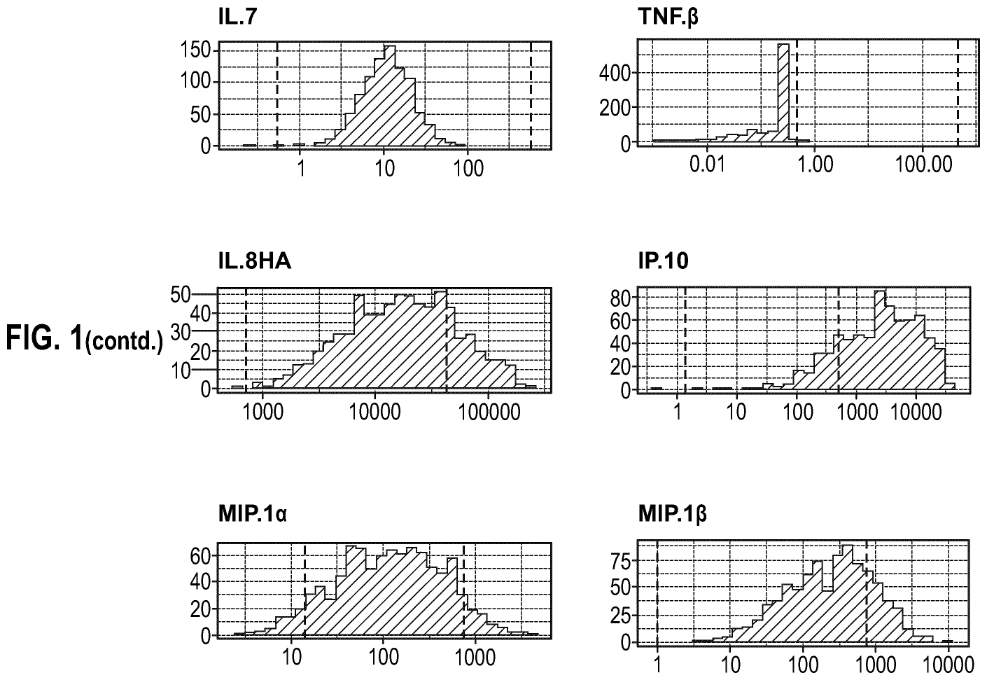

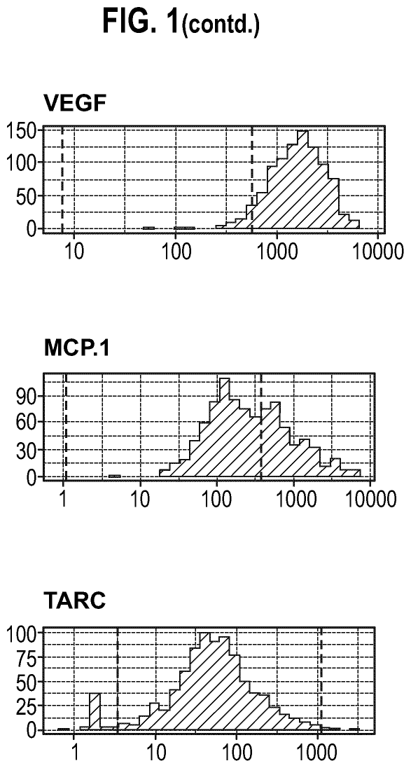

FIG. 1 shows (per each cytokine) the distribution of the raw valid results. Values not respecting the acceptance criteria were excluded from the analysis and classified as NAs (Not Available data).

The acceptance criteria were two-fold
1. Plate Acceptance Criteria
   The Mean relative error (MRE) calculated from the standard curve points was required to be less than 15% (plate accepted). If the MRE was above 15%, it was considered acceptable to remove up to two standard curve points in the upper and/or lower range of detection and to calculate again the M.R.E. %. If after the removal of two selected dilutions M.R.E. %<15%, the plate is accepted. If not, the acceptance criteria were not met.
   The MSD kit provides three different concentration controls for each analyte. The percentage of recovery was calculated directly by the instrument and represented the deviation between the observed and the expected concentration values for each control. In order to accept the run, two out of three MSD® controls required a Percentage of Recovery between 70% and 130% for each cytokine.
2. Sample Acceptance Criteria

To evaluate if an analyte concentration could be quantified, four parameters were considered: i) Lower and Upper Limit of Detection (LLOD and ULOD), automatically calculated by the MSD Discovery Workbench software at every run, and ii) Lower and Upper Limit of Quantification (LLOQ and ULOQ), reported in the MSD V-plex Certificate of Analysis.

The ULOQ and LLOQ are the highest and lowest standard curve points that can still be used for quantification; they are the values below and above which, respectively, quantitative results may be obtained with a specified degree of confidence, or the highest/lowest concentration of an analyte that can be accurately measured. Together, the ULOQ and LLOQ define the range of quantification for the assay.

The ULOQ was the highest concentration at which the CV of the calculated concentration is <20% and the recovery of each analyte is within 80% to 120% of the known value. The LLOQ is the lowest concentration at which the CV of the calculated concentration is <20% and recovery of each analyte is within 80% to 120% of the known value.

The sample acceptance criteria were defined as the following:
The concentration value is considered valid if it is within the quantification range: LLOQ<Value<ULOQ.
Concentration values above the ULOQ or below the LLOQ could not be considered as valid since precision and linearity in these regions were not assessed.

Vertical dashed lines in each subpanel (FIG. 1) represent the Lower Limit of Quantification (LLOQ) and the Upper Limit of Quantification (ULOQ) respectively. For each cytokine, all the values below the LLOQ were set to an arbitrary low value of LLOQ/4 and all the samples above the ULOQ were set to an arbitrary high value of LLOQ*4.

Only CTKs for which at least the 20% of the distribution is within the quantification range were included within the analysis. Consequently IL-8, GM-CSF, TNFβ and VEGF were excluded.

To avoid possible bias, CTKs with a percentage of not valid results (NAs) greater than 8% were excluded. IL-12p40, Eotaxin, Eotaxin3, IL-8HA, IP-10, MCP-4, MDC, MIP-1β and TARC were excluded.

### Example 5: Differentiation of Exacerbated and Stable States

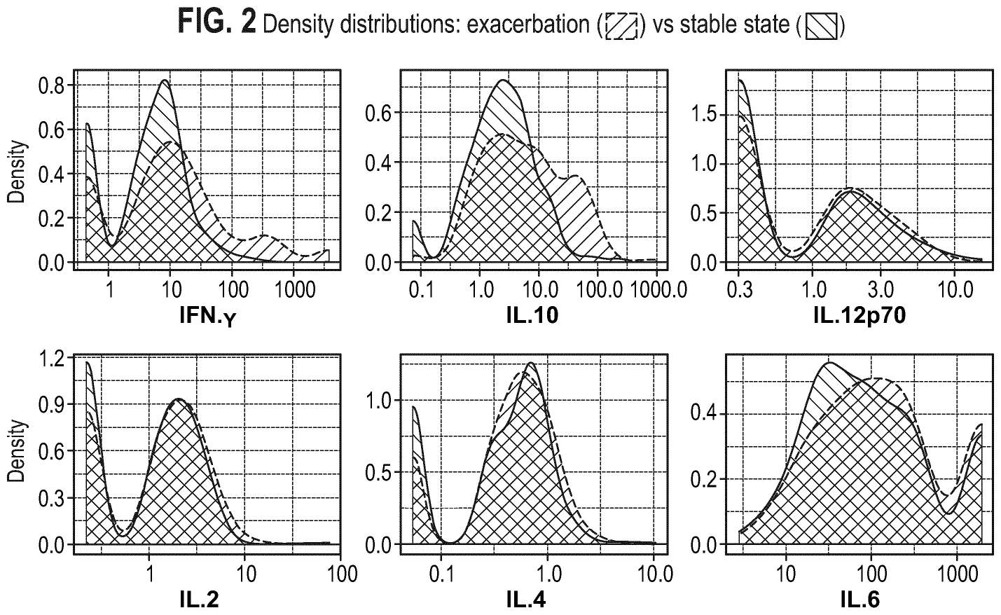

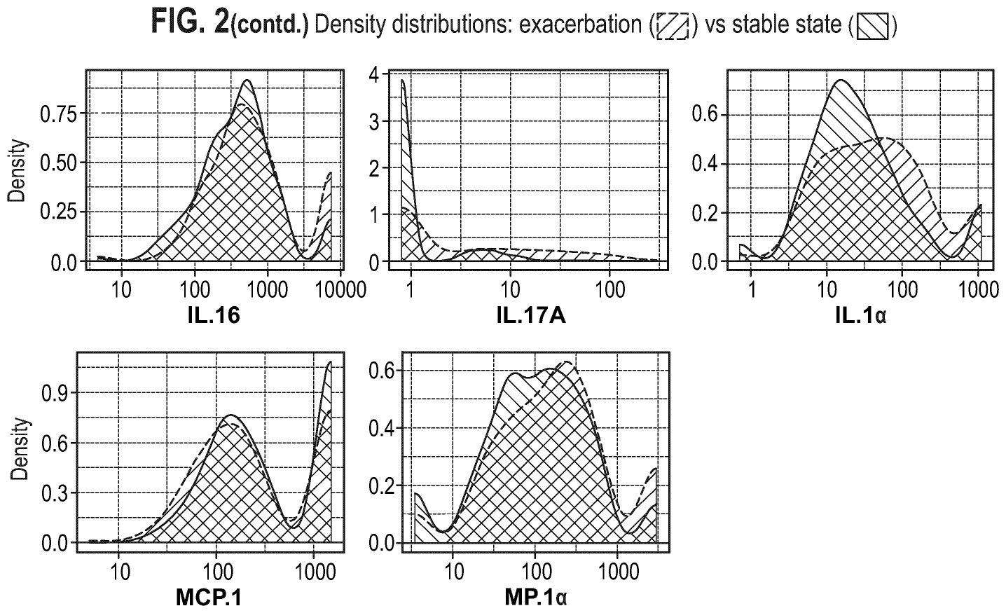

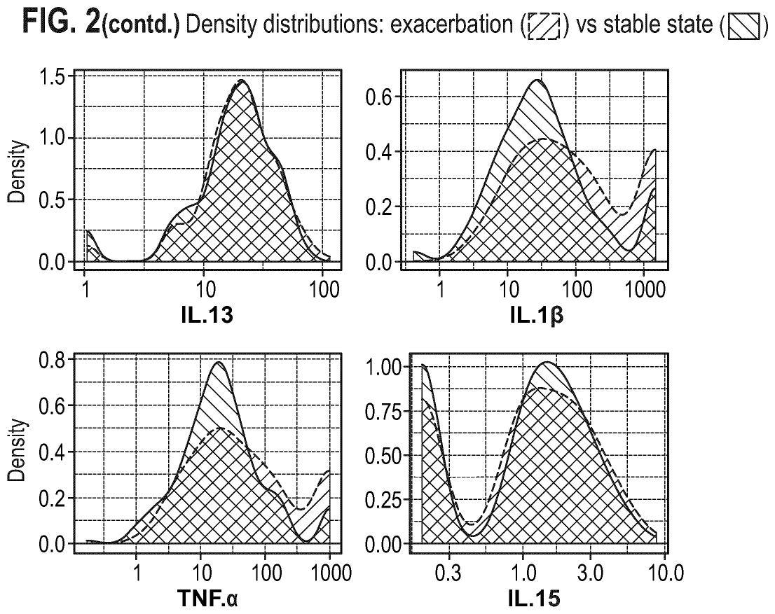

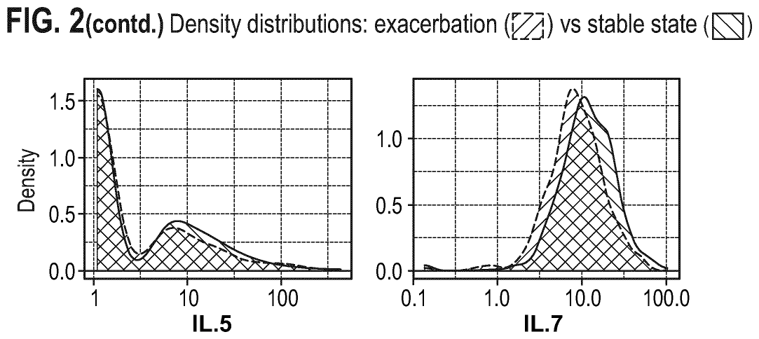

Univariate analysis: For each of the 17 CTKs included in the analysis, FIG. 2 shows the density distribution of the cytokine concentration for stable and exacerbation states, normalized to the same area under the curve.

Since data are not independent (each subject has been tested in multiple visits), for each cytokine i we applied a linear mixed effect model to evaluate differences between groups (ST or EX states):

yᵢ=βₒ+βᵢxᵢ+e eq. 1

The model was fitted on CTK Concentration (yi) including the disease state (xi) as fixed effect and the subject as random effect (c).

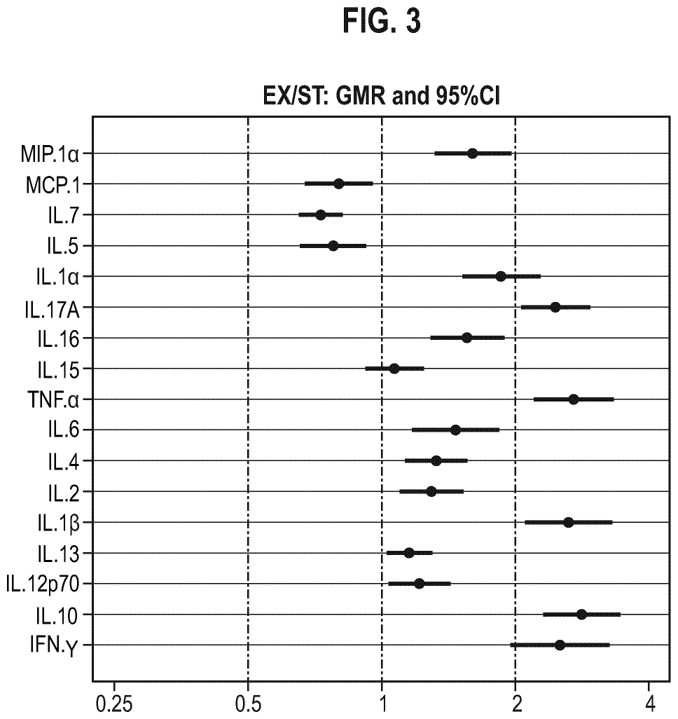

FIG. 3 presents, for each CTK, the results from the mixed model: the bars are the exponential (base 10) 95% CIs of the Estimated Marginal Means (EMMs) in each group (ST or EX).

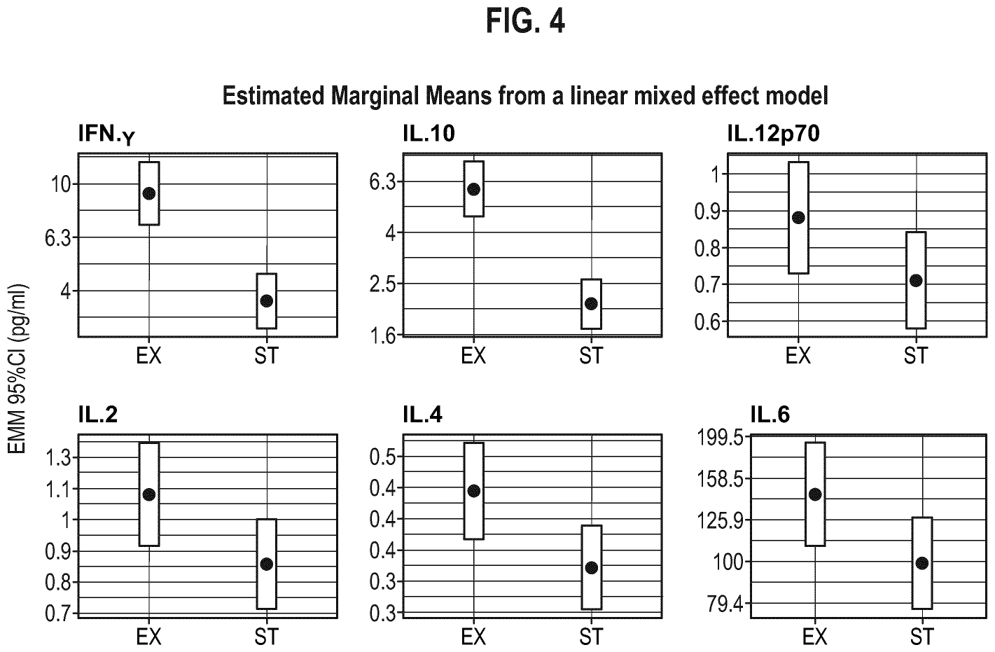

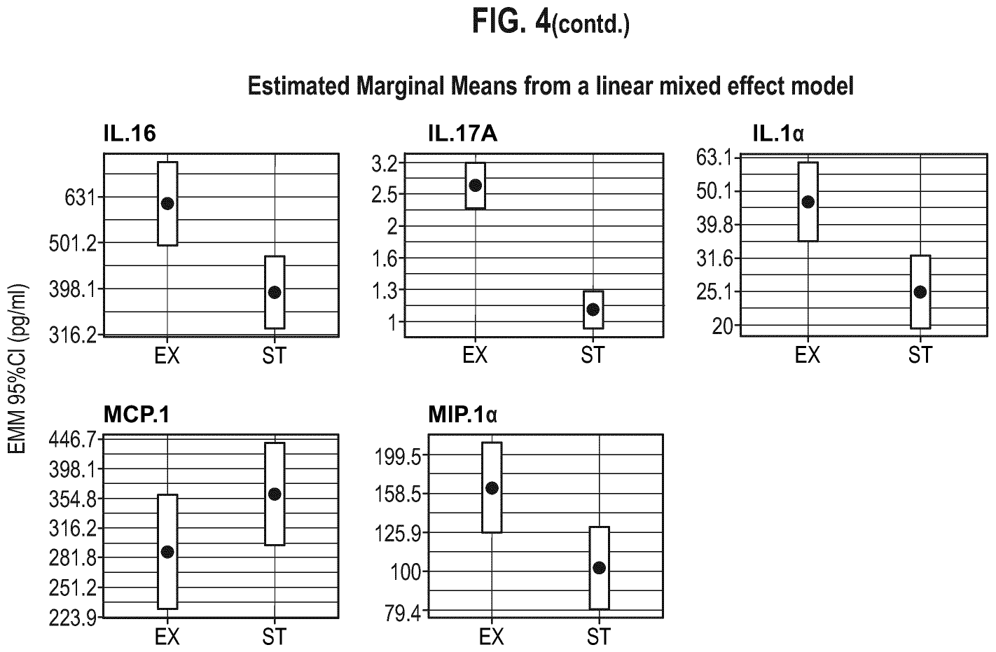

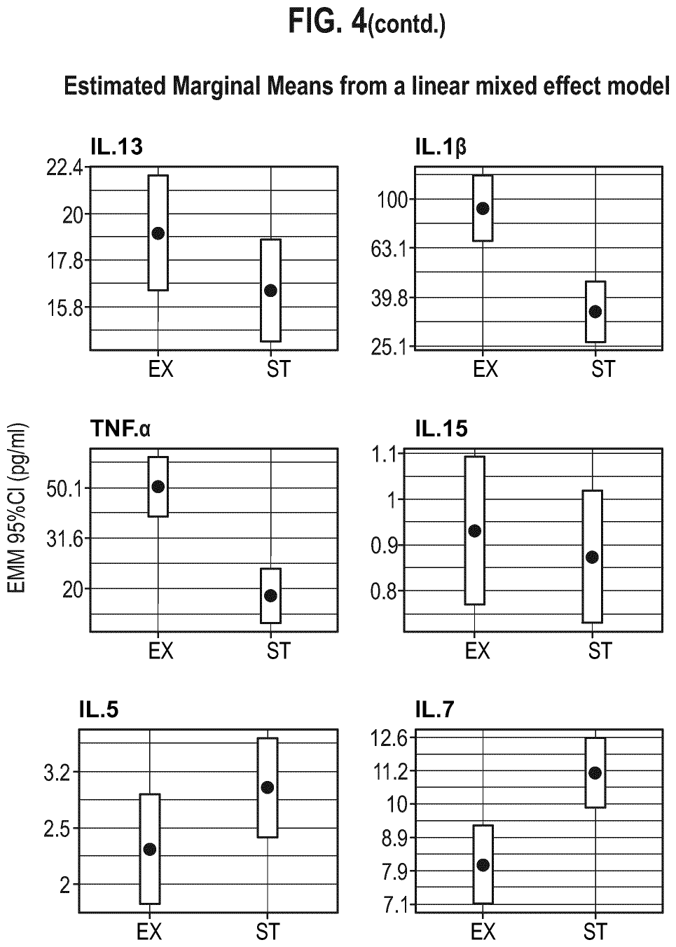

To compare the EMMs between disease states, for each cytokine we calculated the geometric mean ratio (GMRs) and two-sided 95% CIs by exponentiation (base 10) of the contrast of those EMMs. Results, reported in FIG. 4, show that during exacerbations the level of IL-17A, TNF-α, IL-1β and IL-10 significantly increase (>2-fold, red line) compared to the level measured in stable state.

Multivariate analysis: To consider simultaneously the contribution of all CTKs in predicting the disease state, we applied to our data the following logistic mixed model:

y=βo=β₁x₁+ . . . β₁₇x₁₇+β₁₈x₁x₂. . . Bₙxᵢxⱼ. . . +B_{z}x₁₇x₁₆=ε eq. 2

where y is a binary variable equal to 1 if the state is an exacerbation (EX) or equal to 0 if the state is stable (ST). x1 . . . x17 are the CTK concentrations (log 10 transformed and standardized) and β1 . . . β17 are the respective coefficients. ε is the by-subject random effect. Because of the high correlation among variables we excluded the interaction terms from this model.

To evaluate the performance of the model, we randomly split the complete data into training and test sets. Model parameters were estimated from a balanced training set containing the 75% of the complete dataset. To avoid possible bias in parameter estimations, we measured collinearity by calculating a variance inflection factor (VIF2) for each predictor. We excluded from the model in eq. 2 TNF-α and for IL-1β, since they VIF exceeded 5.0 [8].

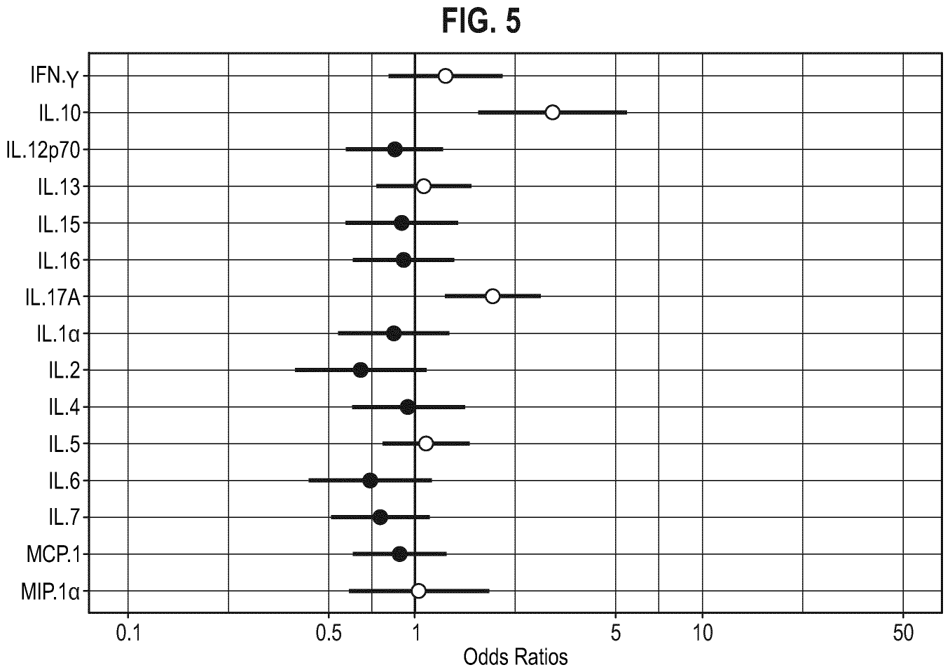

The odds ratios are reported in FIG. 5. Only for IL-10 and IL-17A a significant positive effect exists.

By applying the model on the independent test set, we measured an accuracy of 88%, a specificity of 97% and a low sensitivity of 39%. Therefore, the increased level of cytokines measured in sputum during exacerbation seems to be not enough to predict the state of the disease.

### Example 6: Correlation Between Sputum Cytokines and Type of Infection

Infection types are derived from the described AECOPD types. In our analysis, the following types are also applied to samples taken at stable visits:
Bacterial (B): PCR positive in at least one of Haemophilus influenzae, Streptococcus pneumoniae, Staphylococcus aureus, Moraxella catarrhalis, Pseudomonas aeruginosa in sputum samples.
Viral (V): (q)PCR positive for at least one viral tested on DNA extracted from sputum samples.
Eosinophilic (E)
   Eosinophils are greater than 3% of non-squamous cells from sputum.
   Eosinophils are greater than 2% of cells from WB.
Pauci: not Bacterial, not Viral and not Eosinophilic
Mixed: characterized by a combination of types; BE, BV, BEV, EV.

The “mixed” subgroup was excluded from the analysis

Univariate analysis: To evaluate differences between type of infection (B, V, E and Pauci) associated with state of disease (ST or EX states), for each cytokine i we applied a linear mixed effect model:

yᵢ=αₒ+αᵢxᵢ+βᵢzᵢ+γᵢxᵢzᵢ+ε eq. 3

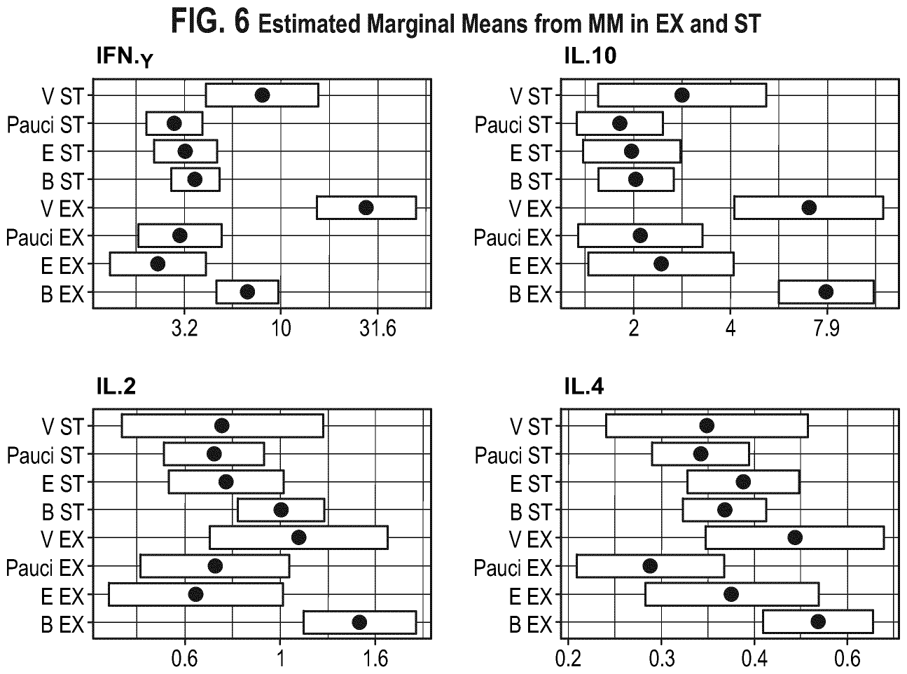

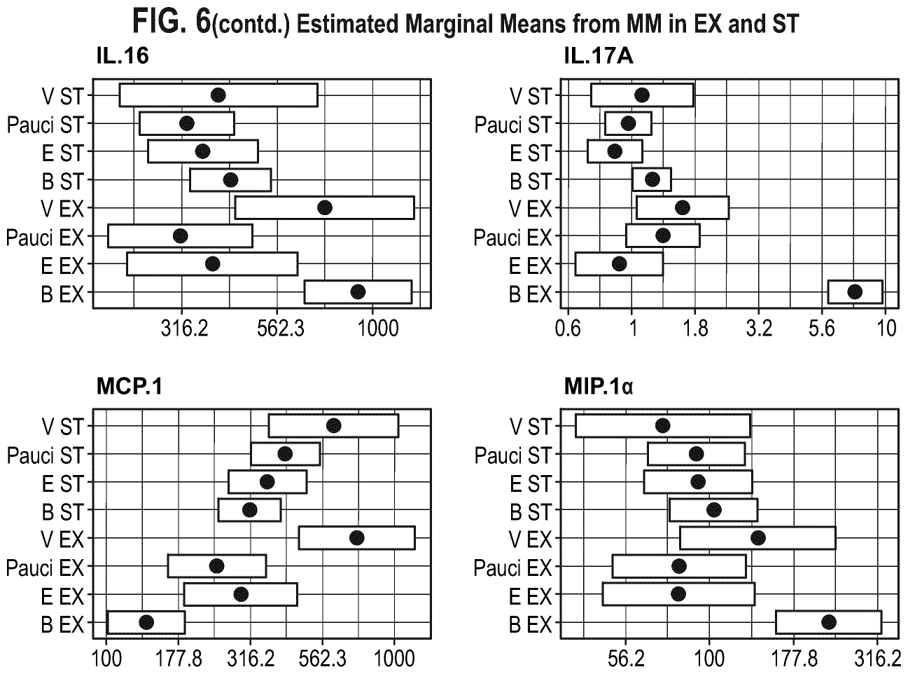

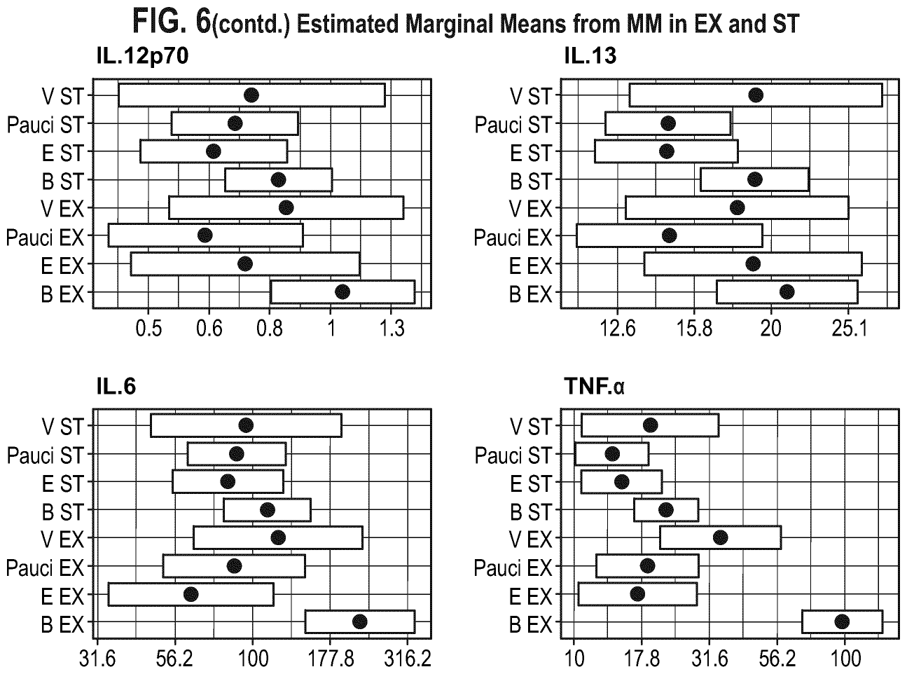

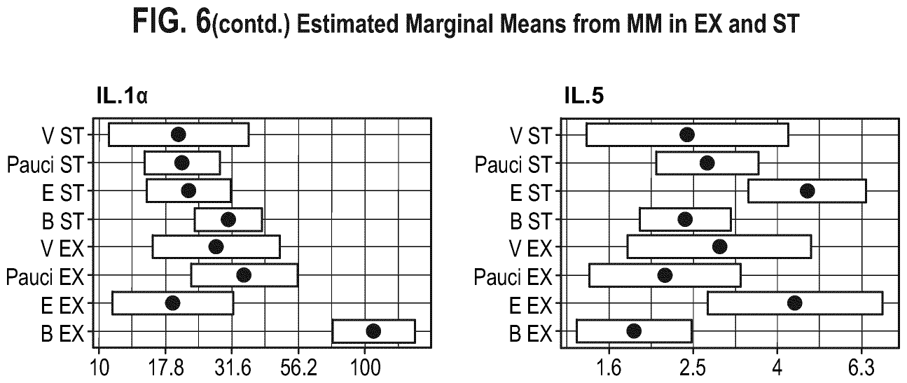

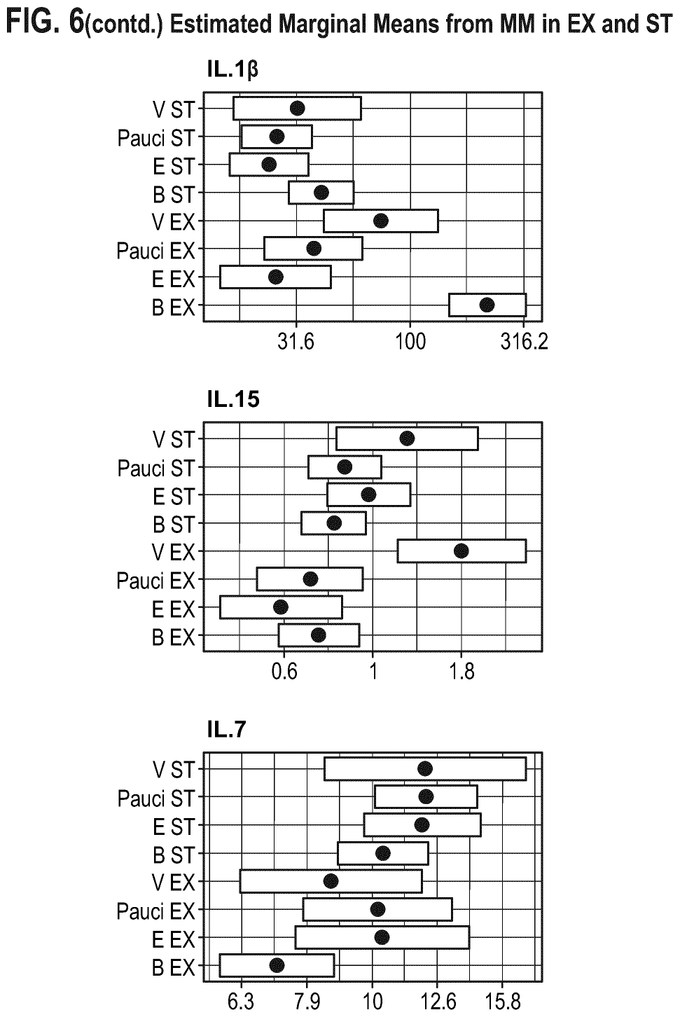

The model was fitted on CTK concentration (yᵢ) including the disease state (xᵢ) and the type of infection (zᵢ) as fixed effect and the subject as random effect (ε). Interaction term (xᵢzᵢ) was considered in the model as well. FIG. 6 presents, for each CTK, the results from the model: the grey bars are the 95% confidence intervals for the Estimated Marginal Means (EMMs) in each group.

A clear and significant separation can be measured only for exacerbations associated with bacterial infections (group B EX in the figure) in IL-17A, TNF-α, IL-1β and IL-1α. No single cytokine differentiates among exacerbations associated with eosinophilia or with viruses from other type of infections (in EX and ST).

Roc characterization: To identify suitable biomarkers for exacerbation associated with bacteria, we applied a ROC characterization. ROC curve is a performance measurement for classification problem at various thresholds settings.

To avoid biases induced by the dependent observations in the complete data (subjects with multiple visits), we apply the ROC characterization to an artificial dataset obtained by selecting randomly an exacerbation sample from each subject, for total of 63 observations.

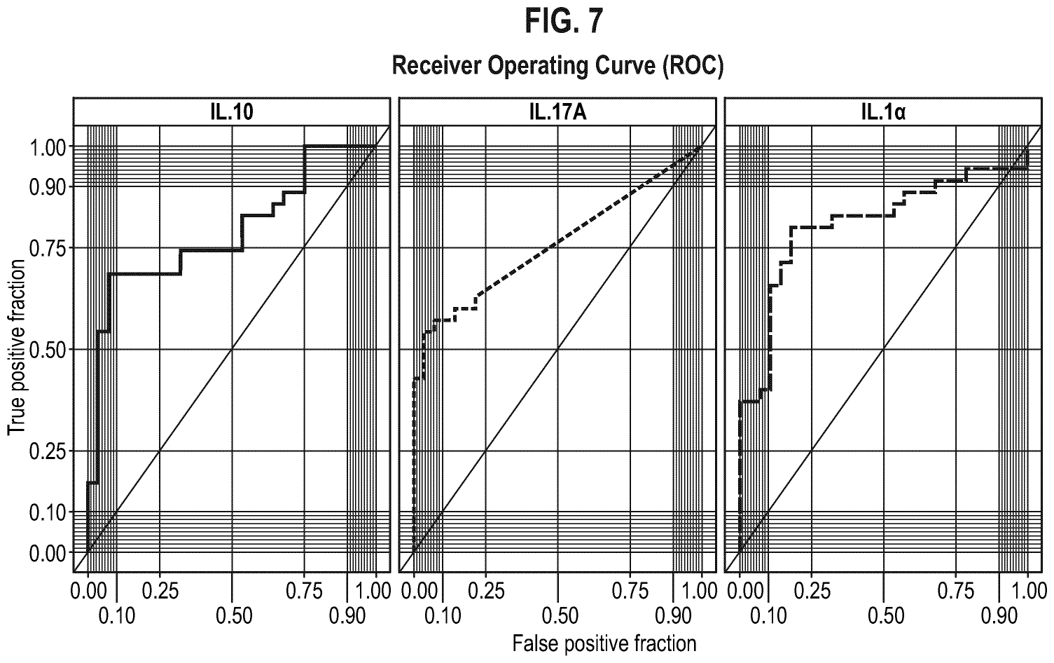

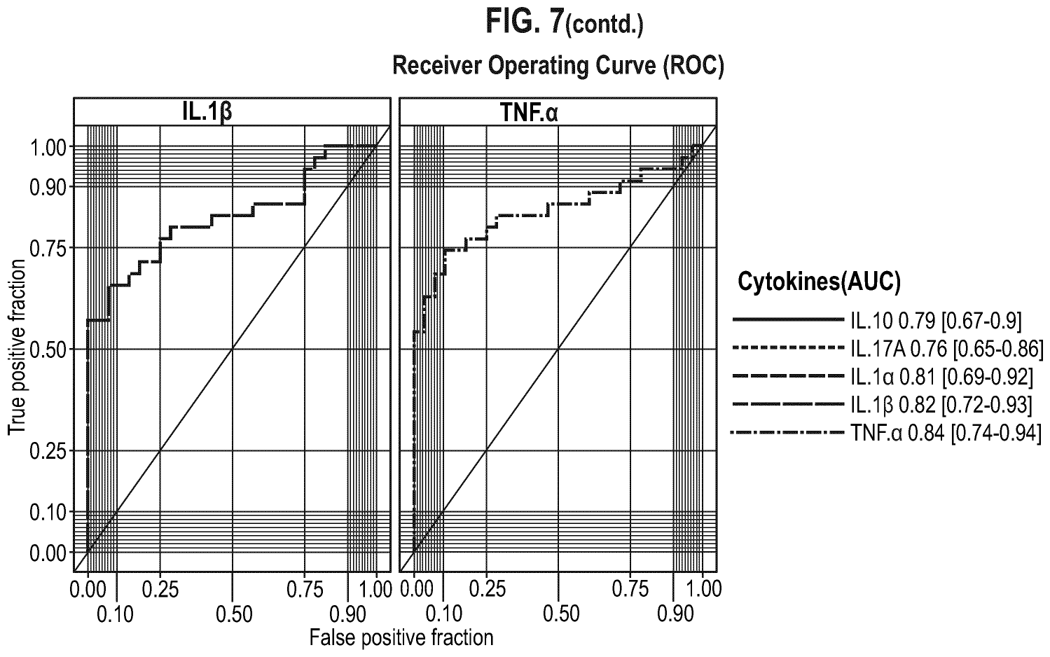

ROC curve has been reported in FIG. 7 only for cytokines for which the AUC is greater the 0.75. The most suitable biomarkers for determining bacteria-associated exacerbation were sputum concentration of IL-1α, IL-1β and TNF-α.

For IL-1α, a threshold of 46 pg/ml had a sensitivity of 80% and a specificity of 82%.

For IL-1β, a threshold of 125 pg/ml had a sensitivity of 66% and a specificity of 93%.

For TNF-α, a threshold of 40 pg/ml had a sensitivity of 74% and a specificity of 89%.

No single biomarker had an AUC>0.7 in determining presence of bacteria in stable state samples (ST).

Further analysis: We evaluated how specificity and sensitivity parameters change if the level of IL-1α, IL-1β and TNF-α are all (or two out of three) above the respective cutoff: 46, 125 and 46 pg/ml. Table 1 reports results from this computation.

| | Sensitivity | Specificity | |
|---|---|---|---|
| | (% of bacterial | (% of no-bacterial | |
| | exacerbation | exacerbation | |
| Biomarker | correctly | correctly | |
| combination | predicted) | predicted) | Accuracy |
| IL.1α + IL.1β | 70% | 97% | 83% |
| IL.1β + TNFα | 70% | 100% | 84% |
| IL.1α + TNFα | 79% | 97% | 87% |
| IL.1α + IL.1β + TNFα | 70% | 100% | 84% |

Then, to better characterize the simultaneous contribution of the three CTKs in predicting bacterial associated exacerbation, we applied to our data the following logistic model (as implemented in the glm R package):

y=βo+β₁x₁+β₂x₂+β₃x₃

y is a binary response equal to 1 if the infection is bacterial or equal to 0 if the infection is not bacterial (Virus, Pauci or eosinophilic). x₁, x₂ and x₃, are the concentration levels (log 10 transformed and standardized) for IL-1α, IL-1β and TNF-α with β₁, β₂ and β₃ as the respective coefficients. β₀ is the intercept. Because of the high correlation among variables we excluded the interaction terms from this model.

To evaluate the performance of the model, we randomly split the dataset into training and test sets. Model parameters were estimated from a balanced training set containing the 70% of the complete dataset. To avoid possible bias in parameter estimations, we measured collinearity by calculating a variance inflection factor (VIF) for each predictor. Since for IL.1β the VIF exceeded 5.0 [2], we included this CTK from the model in Eq.1.

By applying the model on the independent test set, we measured an accuracy of 88%, a specificity of 100% and a sensitivity of 80% in predicting bacteria associated exacerbation. Those parameters, even if estimated from a completely different approach, are consistent with results from those presents in table and confirmed the important role of IL.1α and TNFα.

Overall, from this data if we consider IL.1α, IL.1β and TNFα, the role of IL.1β is redundant. It does not add any value to the IL1α, -TNF-α combination in identifying bacteria associated exacerbations.

### Example 7: Follow-Up Clinical Study

The sputum cytokine analysis that was conducted in Examples 1-6 (AERIS Study) was conducted on a further clinical study.

The analysis presented below aimed to confirm previous results (AERIS Study, Examples 1-6) and describe relationships between cytokines response in sputum samples and type of exacerbations (e.g. bacterial, viral etc).

This analysis formed part of a tertiary objective of the follow-up clinical study. Said tertiary objective was “to collect sputum samples to explore the level of inflammation (into the lung)”

The primary objective meanwhile was to evaluate a clinical stage vaccine candidate, said vaccine candidate being developed to protect subjects against COPD exacerbation (data not shown).

Only the data from patients administered the placebo vaccine is included in this Example (i.e. no data is presented from the arm(s) of the study where patients were administered the candidate COPD vaccine).

Study design: The quantification profiles of 30 cytokines and chemokines measured by Mesoscale in sputum samples, were analysed at Day 1, Day 91, Day 181, Day 271, Day 361, Day 451 and at each AECOPD visit from first vaccination to study conclusion on a subset of COPD patients enrolled in the clinical trial. Said analyses were performed essentially as described in Examples 1-5.

The whole study included 606 subjects split into two approximately equal groups of patients between the candidate vaccine and placebo groups (Table 2). A subset of these patients (“Cytokine Subset”) provided sputum samples at the intervals outlined above. Since the following analysis was only conducted in respect of the placebo group patients, the n-number for the analysis was 87 patients.

TABLE 2

| | Candidate Vaccine | Placebo | Total |
|---|---|---|---|
| | N (%) | N (%) | N (%) |
| Whole Study | 304 (50) | 302 (50) | 606 (100) |
| Cytokine Subset | 83 (49) | 87 (51) | 170 (100) |

Of these 87 patients, ˜30% were stable throughout the whole study, whilst the other ˜70% of patients expressed at least one acute exacerbation of their COPD (table 3).

TABLE 3

| | Placebo |
|---|---|
| | N (%) |
| Cytokine Subset | 87 (100) |
| Stable over the whole study | 25 (29) |
| at least one AECOPD | 62 (71) |

The total number of cytokine samples obtained from the placebo group was 314 (as reported below in Table 4). This is compared to 936 sputum samples in the AERIS Study. However, only 67 out of the 314 total samples (˜21%) were taken during exacerbation episodes.

TABLE 4

| | Placebo |
|---|---|
| | N (%) |
| Cytokine samples | 314 |
| Stable visits with cytokine sample | 247 |
| Exacerbation visits with cytokine sample | 67 (100) |
| MILD | 4 (6) |
| MODERATE | 55 (82) |
| SEVERE | 8 (12) |

Results: Having repeated the uni/multi-variate analysis described in Examples 5-6 it was concluded that, due to the low sample size, the study was insufficiently powered. As such, it was not possible to statistically confirm our previous findings from Example 5 and 6.

This was particularly evident when attempting to identify cytokines that were capable of differentiating an exacerbation that is associated with a bacterial exacerbation from exacerbations associated with, for example, viral exacerbations. For example, only 11 cytokines samples were obtained from patients suffering from exacerbations associated with viral-only infections and only 29 sample were obtained from bacterial only infection (NTHi, Mcat or NTHi+Mcat).

Furthermore, unlike Examples 1-5 (AERIS Study), no data was collected relating to eosinophilic infections (i.e. not bacterial, viral). Therefore, an eosinophilic component to the exacerbation types measured in this study cannot be ruled out.

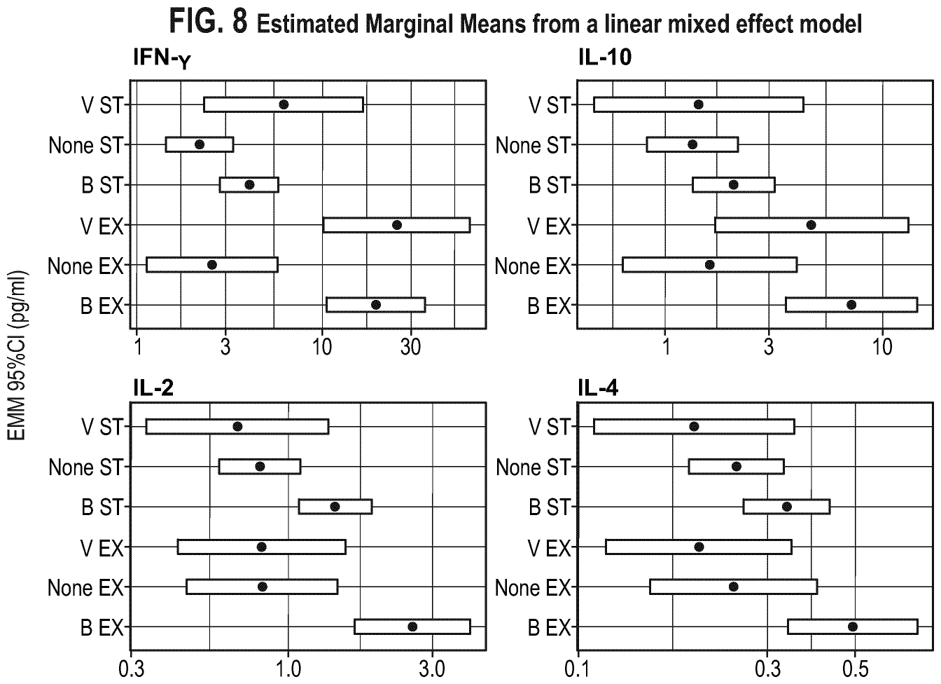

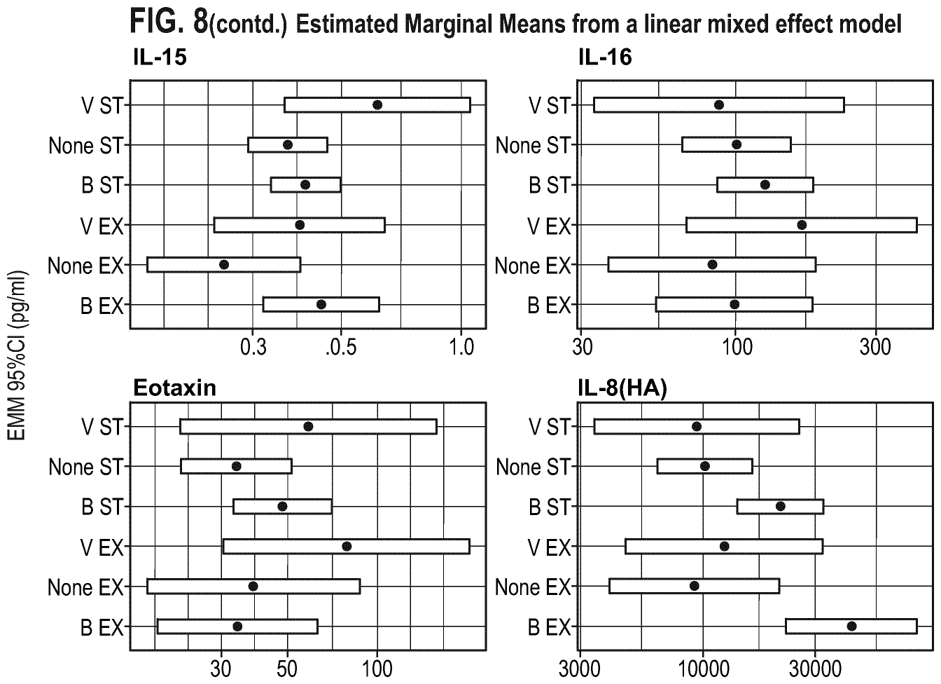

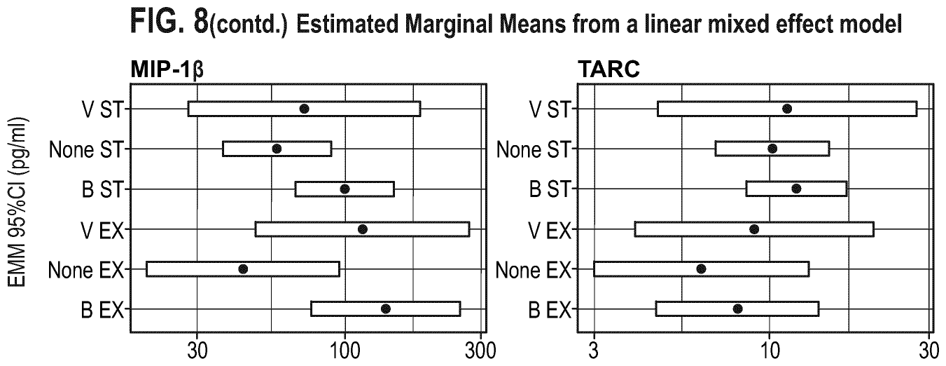

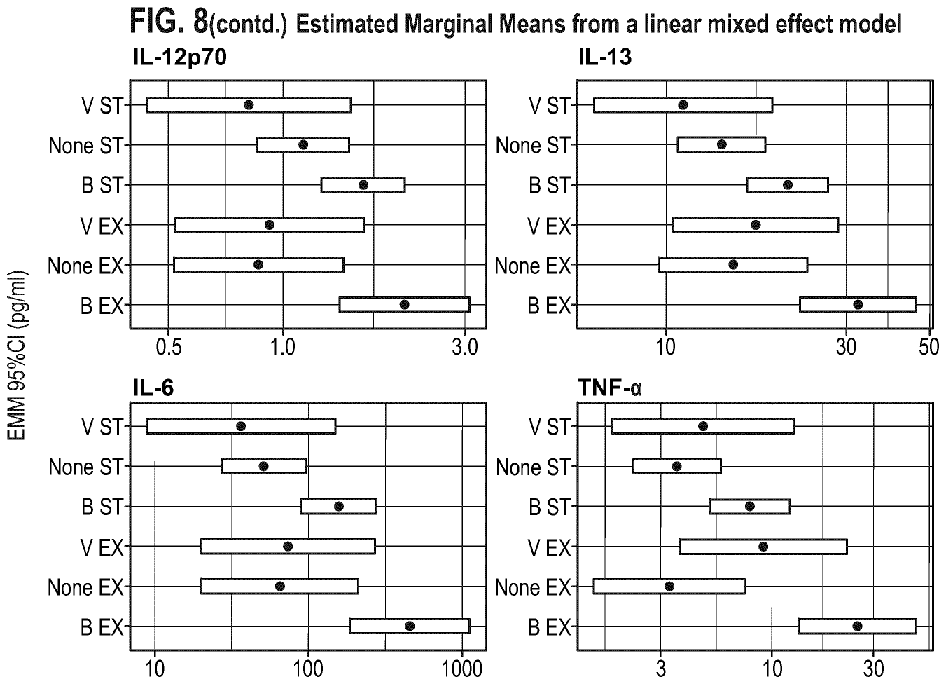

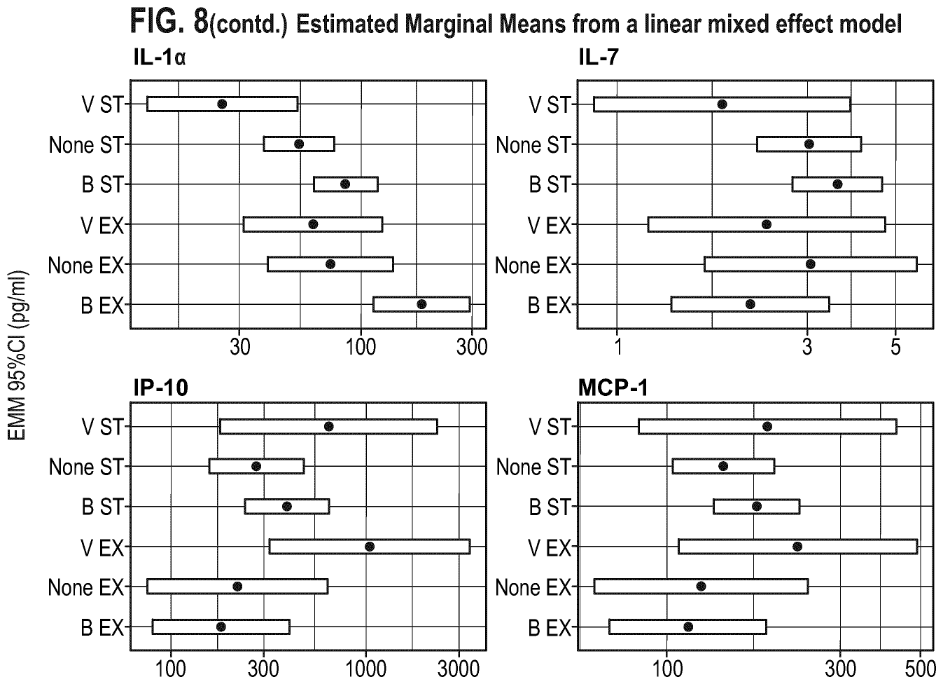

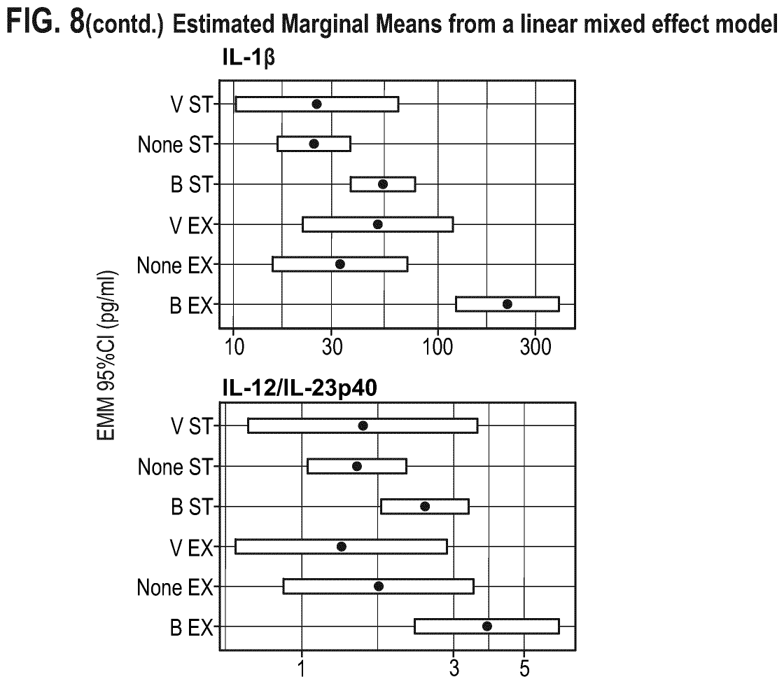

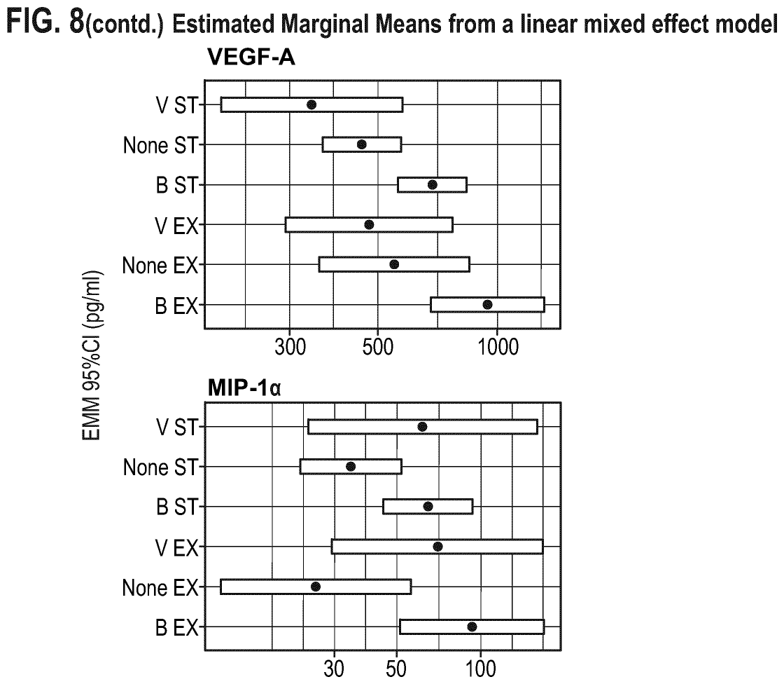

Nonetheless, despite greater variability compared to the same analysis from the AERIS study (see FIG. 6), the data shown in FIG. 8 shows a trend which confirms that the concentration of IL1-alpha and TNF-alpha are increased in subjects that are experiencing a bacterial exacerbation (B EX in FIG. 8).

1. (canceled)

2. (canceled)

3. A method of treating an exacerbation of chronic obstructive pulmonary disease (COPD) in a subject comprising the steps of:
a) measuring the concentration of IL-1α and TNF-α in a biological sample obtained from the subject,
b) detecting that the concentration of IL-1α and TNF-α are increased relative to a reference biological sample,
c) differentiating that the subject is suffering from an exacerbation of COPD that is associated with a bacterial infection, and
d) administering a therapeutically effective amount of an antibiotic agent to the subject.

4. (canceled)

5. (canceled)

6. (canceled)

7. (canceled)

8. The method of claim 3, wherein the antibiotic agent is of the beta-lactam, macrolide, quinolone, cephalosporin, aminoglycoside or tetracycline class of antibiotic.

9. The method of claim 3, wherein the antibiotic agent is doxycycline, trimethoprim-sulfamethoxazole, amoxicillin-clavulanate potassium, clarithromycin, azithromycin, levofloxacin, gatifloxacin, moxifloxacin, penicillin, ampicillin, amoxicillin, cefaclor, cefuroxime, cefdinir, cefixime, ceftriaxone, cefotaxime, ceftazidime, cefepime, piperacillin-tazobactam, ticarcillin-clavulanate potassium, levofloxacin/moxifloxacin, gatifloxacin and/or tobramycin.

10. The method of claim 3, wherein the biological sample is a sputum sample.

11. The method of claim 3, wherein the subject is suffering from an exacerbation of COPD that is associated with a bacterial infection rather than an exacerbation that is associated with a viral or eosinophilic infection, or a pauci exacerbation.

12. The method of claim 3, wherein the concentration of IL-1α is ≥46 pg/mL and the concentration of TNF-α is ≥40 pg/mL in the biological sample.

13. The method of claim 3, wherein the concentration of IL-1α and TNF-α in the biological sample is higher than the concentration of IL-1α and TNF-α in the reference biological sample.

14. The method according to claim 13, wherein the concentration of IL-1α and TNF-α α in the biological sample is greater than 2-fold, greater than 3-fold, greater than 4-fold, greater than 5-fold, greater than 10-fold, greater than 25-fold or greater than 50-fold higher than the concentration of IL-1α and TNF-α in the reference biological sample.

15. The method of claim 13, wherein the reference biological sample is either (i) a biological sample obtained from a healthy subject without COPD or (ii) a biological sample obtained from a subject with COPD but who does not have an exacerbation.

16. The method of claim 3, wherein the subject is a human.

17. The method of claim 3, wherein the concentration of IL-1α and TNF-α are measured by an immunoassay.

18. The method according to claim 17, wherein the immunoassay is an ELISA based assay.

19. (canceled)

20. The method of claim 3, wherein the bacterial infection is an infection that comprises at least one of Haemophilus influenzae, Streptococcus pneumoniae, Streptococcus pyogenes, Staphylococcus aureus, Moraxella catarrhalis and/or Pseudomonas aeruginosa.

21. The method of claim 1, wherein the subject is not administered systemic corticosteroid-based therapy.

22. The method according to claim 13, wherein the biological sample is a sputum sample.

23. The method according to claim 13, wherein the reference biological sample is a sputum sample.
